SEQUENCE LISTING (12 sequences)

SEQ ID NO: 1 - protein, 4 aa, Artificial Sequence
  common linker motif found in natural proteins
GGGS

SEQ ID NO: 2 - protein, 15 aa, Artificial Sequence
  linker motif peptide between the antibody and the neurotrophic molecule part
GGGGSGGGGSGGGGS

SEQ ID NO: 3 - DNA, 81 bp, Artificial Sequence
  Primer AB 176-S, 5′ primer that carries HindIII, SalI, and AgeI sites, used in the amplification of the Fd chain of IN-1
agaaaagtgaaatggaagcttcgacaaaattctgtcgaccgagggcaaaaaaccggtgggggaggttccgggggtggaggg

SEQ ID NO: 4 - DNA, 100 bp, Artificial Sequence
  Primer AB 239-S, 5′ primer that carries a XhoI restriction site, used in the amplification of the Fd chain of IN-1
gggaggttccgggggtggagggtcagggggtggtggctctggcggaggggggtccgaagttaaactgctcgagtcagggcctgggctggtaaggcctggg

SEQ ID NO: 5 - DNA, 25 bp, Artificial Sequence
  Primer AB 912-AS, 3′ primer used in the amplification of the Fd chain of IN-1
ttctgctagcttattaatggtgatg

SEQ ID NO: 6 - DNA, 22 bp, Artificial Sequence
  Primer AB 996-S, primer used in the PCR amplification the light chain IN-1
aaaagccgacatcgagctcacc

SEQ ID NO: 7 - DNA, 30 bp, Artificial Sequence
  Primer AB 1647-AS, primer containing an XbaI site, used in the PCR amplification of the light chain of IN-1
gcagcatcaggacctctagaactaacactc

SEQ ID NO: 8 - DNA, 22 bp, Artificial Sequence
  Primer 169-AS, primer used for nucleic acid sequencing on the 5′ end of the NT-3 cDNA
gggaattgagagagtcttctgg

SEQ ID NO: 9 - DNA, 22 bp, Artificial Sequence
  Primer 774-S, primer used for nucleic acid sequencing on the 5′ end of the NT-3 cDNA
gcaaacctacgtccgagcactg

SEQ ID NO: 10 - DNA, 34 bp, Artificial Sequence
  Primer 881-S, hybridizing primer used in the mutagenesis of the existing stop codon in NT-3
gaaaaatcggaagaaccggtattggcatctgtcc

SEQ ID NO: 11 - DNA, 34 bp, Artificial Sequence
  Primer 848-As, hybridizing primer used in the mutagenesis of the existing stop codon of NT-3
ggacagatgccaataccggttcttccgatttttc

SEQ ID NO: 12 - DNA, 28 bp, Artificial Sequence
  Primer 96-S, used in the PCR amplification of NT-3 cDNA
gatcttacaggtgaacctcgagatgtcc

(19) United States
(12) Patent Application Publication
(10) Pub. No.: US 2008/0241157 A1
(43) Pub. Date: Oct. 2, 2008

(54) Nerve cell growth modulators (amphibodies)

(76) Inventor: Matt Fraidakis, Athens (GR)

Correspondence Address: WENDEROTH, LIND & PONACK, L.L.P., 2033 K STREET N. W., SUITE 800, WASHINGTON, DC, 20006-1021, US

(21) Appl. No.: 11/710,541

(22) Filed: Feb. 26, 2007

Related U.S. Application Data
(63) Continuation of application No. 09/959,200, filed on Oct. 19, 2001, now abandoned.

(30) Foreign Application Priority Data
Apr. 21, 1999 (SE) 9901428-4

(51) Int. Cl. A61K 39/395; A61K 38/16; A61K 31/715; C12P 21/00; C12N 15/64; C12N 5/08; A61K 38/18; A61K 38/19; A61K 38/22; A61P 25/00; A61P 43/00

(52) U.S. Cl. 424/141.1; 514/8; 514/54; 514/12; 435/69.1; 435/320.1; 435/366

(57) ABSTRACT

The present invention relates to a nerve cell growth modulator denoted an amphibody. The amphibody according to the invention is comprised of at least two components, wherein a first component is capable of binding to a target and suppressing, or essentially neutralising, a nerve cell growth inhibitory effect thereof; and a second component is capable of stimulating nerve cell growth and/or regeneration. In the most preferred embodiment, the present amphibody is a neuromodulator comprised of the above disclosed two components connected by a linker element. Amphibodies according to the invention are a useful means of molecular disguise of a non-permissive environment into a permissive, and outgrowth promotive and favorable one. The present invention also relates to genetic and chemical methods of preparation of an amphibody according to the invention as well as to various advantageous uses thereof.

This application is a continuation of Ser. No. 09/959,200, filed Oct. 19, 2001, now abandoned.

### TECHNICAL FIELD

The present invention relates to novel bipartite compounds useful in enhancing the growth and regeneration of cells, including neurons and nerve fibres. The invention also relates to methods of producing the novel compounds as well as to various pharmaceutical uses thereof.

### BACKGROUND

Regeneration of cells is a prerequisite for tissue repair in all eucaryotic life forms. In mammals, regulation and modulation of cell regeneration is often governed by a complex interaction of stimulatory and inhibitory factors.

One cell type, whose growth and regeneration would be advantageous to control, is the nerve cell and its neurites. The mammalian nervous system is divided into two parts, the peripheral nervous system (PNS), comprising the peripheral nerves and ganglia, and the central nervous system (CNS), comprising the brain and spinal cord. When nerve fibers are injured in mammals including man, the peripheral stump which is no longer connected to the nerve cell body will always degenerate. Recovery of function necessitates regeneration by outgrowth of a new nerve fiber from the proximal stump and reestablishment of anatomically correct and functional synaptic connections. In the peripheral nervous system, there is capacity for spontaneous axonal regeneration, particularly if the proximal stump is brought into contact with the remaining ensheathing tissues of the peripheral stump. The glial cells that normally support nerve fibers in the PNS, the so-called Schwann cells, actively secrete neurite growth-stimulating proteins to stimulate regeneration and are very effective in migrating and remyelinating regrowing and regenerating axons. Still, many neurons simply do not survive the insult of axotomy in the PNS.

In the mammalian central nervous system, the capacity for spontaneous regeneration is additionally hampered compared to the PNS. The glial cells ensheathing nerve fibers in the central nervous system, the oligodendroglial cells, actively inhibit regeneration by expressing neurite growth inhibitory molecules on their surfaces, are poor providers of neurotrophic factors and are not apt in initiating remyelination. Other CNS macroglial cells, the so called astrocytes, proliferate locally to form a scar that acts as a barrier to any spontaneously regenerating axons, both physically and chemically through secretion of inhibitory extracellular matrix elements. The proximal part of the severed axons suffers demyelination in the deleterious process of secondary injury, that ensues the merely mechanical primary injury. The proliferating astrocytes thereupon may impede the remyelination of regenerating neurites.

Furthermore, bloodborne inflammatory cells and fibroblasts infuse the lesion site, causing further injury and aggravating the local scar. During the long life of a human being, the likelihood that disease, trauma, and other forms of physical or chemical stress will cause damage to nerve fibers in the brain or spinal cord is substantial. When extensive, such damage will lead to life-long physical or mental handicap. In many such instances, treatment strategies aimed at inducing nerve fiber growth and regeneration and synapse formation in the brain and spinal cord or the peripheral nervous system would prove beneficial.

### PRIOR ART

WO 95/13291 relates to the use of neuron-glia CAMs (Ng-CAM) in the treatment of nerve damage. More specifically, it has been shown that cell adhesion molecules such as Ng-CAM are capable of promoting neuronal adhesion, neutralizing inhibitory effects of extracellular matrix molecules and providing stimulatory signals to neurons. The properties observed according to WO 95/13291 are the same natural properties and pathways of a body after a nerve injury, even though normally, they appear in a delayed manner. Thus, the teachings of WO 95/13291 are to instead introduce said molecules sooner after injury, and thereby accelarate the rate and extent of the natural recovery. Further, WO 95/13291 also suggests to administer Ng-CAM in combination with another well known nerve growth promoting agent, such as nerve growth factor (NGF).

WO 94/17831 relates to a method of promoting central nervous system regeneration by administration of a neurotrophin family member and an antibody to a neurite growth regulatory factor administered together or separately by different routes.

U.S. Pat. No. 5,684,133, in the name of Schwab et al., describe a gene and the encoded protein which functions as neurite growth inhibitory factor. Further, antibodies against such proteins are disclosed. Thus, the teaching of Schwab et al enables the blocking of one out of an expanding number of neurite growth inhibitors with documented function. Apart from not being the sole suppressor of regeneration, there is increasing evidence that long-distance nerve fiber growth can occur post injury in the CNS white matter, despite its abundance of myelin-associated inhibitor (as described in Schwab et al., supra). Furthermore, in the mammalian CNS, sufficient growth stimulatory support is not offered, contrary to the PNS, where the Schwann-cells provide such an effect. Thus, a treatment scheme according to Schwab et al of a condition in the CNS of a mammal does not address all inhibitory elements of the traumatized CNS and, secondly and does not improve on the paucity of stimulatory factors in the CNS environment. In this field, there is a need of a method and means to induce and improve tissue repair in the mammalian nervous system by effectively addressing both the regional inhibitory properties and paucity of stimulatory signals to the benefit of regenerating nerve fibers. This therapeutic principle can have broader applications in the repair of non-neural tissues.

### SUMMARY OF THE INVENTION

The object of the present invention is to fulfill the need defined above by enabling the efficient conversion of a given inhibitory biological environment into a stimulatory one. This is achieved by a new bipartite molecule, which is capable of directing nerve fibre growth to specified cell surfaces or othe components of a tissue.

More specifically, the present invention relates to a neuromodulator comprising at least two components, wherein a first component is capable of binding to a target and suppressing, or essentially neutralising, a neurite growth inhibitory effect thereof; and a second component is capable of stimulating neurite growth and/or regeneration. Thus, this embodiment enables the conversion of a neurite outgrowth- and/or regrowth-inhibitory CNS or PNS environment into a neurite growth stimulatory one. This is accomplished by the manufacture and application of a bipartite compound herein denoted an amphibody, wherein one part of the molecule, the anti-end, is capable of locating, binding to and neutralising, i.e. dock on and cap, inhibitory molecules in the tissue environment, while the other part of the amphibody, the pro-end, will simultaneously and in situ present a neurite growth stimulating substance to the regenerating axon in the place of the previously exposed inhibitory site. Thus, the present amphibodies may be regarded as molecular magnets wherein bipolar features have been fused together in a bidirectional configuration.

Amphibodies according to the invention are a useful means of molecular disguise of, and even force, a non-permissive, or outgrowth-suppressive or chemorepulsive, i.e. an unfavorable environment, encompassing cellular surfaces, extracellular matrix, molecules in the extracellular fluid, into a permissive, and outgrowth promotive and chemoattractive, i.e. a favorable, one. This is accomplished by establishing a pro-growth/pro-regeneration interface on the preexisting unfavourable biological surface, as explained in more detail below.

Thus, the novel combination of the above disclosed first and second components into a single modulating molecule will provide unique advantages by providing a specific and localised simultaneous inhibitory and stimulatory action, which cannot be obtained by the administration of the two components as such.

### BRIEF DESCRIPTION OF THE DRAWINGS

FIG. 1 shows the principle of the new neuromodulator or amphibody according to the invention.

FIG. 2 is a schematic illustration of the function of the amphibodies according to the invention.

FIG. 3 shows three major parts of an exemplary amphibody according to the invention: the antibody, the neurotrophic factor, and a linker in between the two former ones.

FIG. 4 shows how single chain constructs may include both the variable domains (VH and VL) linked via a peptide linker to form a continuous antigen binding molecule, a so called scFv.

FIG. 5 shows the principles of overlapping PCR in the construction of the genes coding for the amphibodies according to the invention.

### DETAILED DESCRIPTION OF THE INVENTION

More specifically, in a first aspect, the present invention in the preferred embodiment relates to a neuromodulator comprising at least two components, wherein

a first component is capable of binding to a target cell or site and of essentially neutralising, or suppressing a neurite growth inhibitory effect thereof;

and a second component is capable of promoting or stimulating neurite regeneration and elongation.

Contrary to known agents for promoting nerve growth, the present invention relates to a single neuromodulator comprised of at least two components of different origin.

Thus, different advantageous properties have been combined into a single dosage form specifically designed for each intended use. The present invention enables for the first time a proportional administration of a designed compound capable of exerting its effect in a specific and predetermined location. Accordingly, the present invention differs from the above discussed WO 95/13291 in that it constitutes a non-naturally occuring combination. Further, the advantageous choice of the nature of the first and second component of the present neuromodulator will result in a compound capable of exerting effects superior to those of Ng-CAM, even if combined with a nerve growth factor. The latter since said nerve growth factor in accordance with WO 95/13291 will have a general and widespread effect in the area, while the second component of the present neuromodulator will be directed to the exact location of nerve growth due to its coupling with the targeting first component.

Of similar reasons, promoting nerve generation using the neuromodulator according to the present invention also differs essentially from WO 94/17831 discussed above, wherein it is suggested to administer a neurotrophin family member and an antibody raised against a neurite growth inhibitory protein, since the effect of said neurotrophin family member will be non-specific instead of localized to act in the close proximity of the nerve growth, where the suppression of the neurite growth inhibitor is provided according to the invention.

Preferably, the present neuromodulators also comprise a linker element, which separates the first and second component to provide a suitable stereometry. As discussed above in the section “Summary of the invention”, the stereochemistry of the present neuromodulator is an essential feature, which clearly distinguish its function from the prior art administration of separate molecules, since it enables molecular disguise of unfavourable conditions at the same time, and in close proximity of, the enhancement of favourable conditions. Thus, the present neuromodulators can be regarded as “molecular magnets” on neuronal axons thanks to their bipartite structure, bidirectional configuration and “bipolar” features. The nature of the linker will depend on the first and second component and the skilled in this field could for each case decide on a suitable separation. In this context, the method of preparation, whether it is by chemical synthesis or recombinant DNA technique, will also be considered when the linker is designed.

Thus, the present invention avoids the stochastic or random axonal growth that the previously known non-specific coadministration methods may cause. Instead, the present neuromodulators put the injured axons back on track by providing guidance and enforcing direction of their growth and/or elongation. A further advantage of the present neuromodulator is that its specific action also enables administration of a smaller amount of theraputic substance.

In a first embodiment, the present neuromodulator is capable of exerting its dual functions in the central nervous system (CNS) of a mammal. In an alternative or additional embodiment, the present neuromodulator is capable of exerting its dual functions in the peripheral nervous system (PNS) of a mammal. Preferably, said functions are exerted in adult mammals, such as adult humans.

In one embodiment of the present neuromodulator, the above mentioned target of the first component is a glial cell, a neuron, a fibroblast, a blood cell or an extracellular matrix component, which provides an inhibitory effect by expressing a specific neurite growth inhibitory molecule. In one specific embodiment of the present neuromodulator, the above mentioned target is an oligodendroglial cell, which provides an inhibitory effect by expressing the neurite growth inhibitory molecule Nogo. In alternative embodiments, said neurite growth inhibitory effect is provided by other inhibitory molecules: myelin associated glycoprotein (MAG); arretin; a proteoglycan; a Sem-receptor; and a member of any one of the families of semaphorins; tenascins, netrins and Eph and ephrins.

In one specific embodiment the first component of the present neuromodulator is IN-1, an antibody against Nogo. These amphibodies may may carry as their second component neurite growth promoting factors in order to rescue neurons and stimulate their fiber regeneration.

The second component of the present neuromodulator may be a neurotropic and/or a neurotrophic molecule. Thus, in one embodiment, the second component of the present neuromodulator is a neurotropic molecule, such as (1) a cell adhesion molecule (CAM), e.g. an immunoglobulin superfamily CAM, a cadherin or an integrin, or any functional fragment thereof, or (2) an extracellular matrix molecule (ECM), such as laminin, a netrin, a tenascin or any functional fragment thereof. In the alternative embodiment, the second component of the present neuromodulator is a neurotrophic molecule selected from the (1) neurotrophin family consisting of nerve growth factor (NGF), brain-derived neurotrophic factor (BDNF), neurotropin 3 (NT3) or neurotropin 4 (NT4), (2) a member of the GDNF-subfamily, preferably glial cell derived neurotrophic factor (GDNF) or its relatives neurturin (NTN), persephin (PNP) or artemin (Art), (3) a neuropoietic cytokine, notably ciliary neurotrophic factor (CNTF), leukemia inhibitory factor (LIF) or cardiotrophin-1 (CT-1), any one of the fibroblast growth factors (FGF), hepatocyte growth factor (HGF) or other factors with documented neuroprotective and stimulatory effects.

The molecules useful in the production of the present neuromodulators, or amphibodies, are preferably chosen among substances having well-documented actions as positive or negative neurite growth regulators implicated in neurohistogenesis and/or influencing the outcome of nervous system injuries. Examples of preferred molecules will be given below under separate headlines for the first component and the second component, respectively. It is apparent to the skilled that such molecules will not all be of similar utility or equal significance for the manufacture and benefits of amphibody applications. However, the skilled in this field will be capable of making a suitable choice among the herein examplified molecules for each separate case, depending upon the specific use and prevailing conditions. In this context, it is noteworthy that the field of axon pathfinding and neurotrophic factors is a booming area of constant discovery (see e.g. Slit (Kuan Hong Wang et al Cell, Vol 96, 771-784, Mar. 19, 1999; Robo receptor (Thomas Kidd, 785-794/Katja Brosek, 795-806/Hua-shun Li 807-818); and Arretin, Monoclonal antibodies against the myelin-derived axon growth inhibitor arretin, R. Janani et al Soc. Neurosci. Abstr., Vol. 24 Part 2, p. 1560, 1998) and increasing complexity, with new molecules yet to be identified new actions to be revealed and old to be redefined.

Consequently, the scope of the present invention is in no way intended to be limited to the molecules exemplified and listed herein. Rather, the gist and the advantages of the present invention can be summarized as follows:

(1) the notion of a novel modulatory molecule, termed amphibody, with simultaneous disinhibiting and stimulatory actions, both contributing to a desired end,

(2) the novel concept of biological or tissue regulation, termed molecular guising, made feasible by the amphibody fulfilling an advantageous dual regulatory capacity as purported above (in (1)),

(3) and any therapeutic implications thereby.

Thus, the list of potential therapeutic agents and any therapeutic implications thereby as presented below should be construed as an illustration only.

In a second aspect, the present invention relates to a general method of producing a neuromodulator comprising the steps of providing a suitable inhibitor or suppressor of a neurite growth inhibiting effect; and providing a molecule with neurotrophic and/or neurotropic properties; and combining the above described components into the desired neuromodulator. Thus, a bipartite neuromodulator is obtained, which enables the molecular guising of a neurite-growth inhibitory or suppressive environment into a neurite growth stimulatory one.

The said neurite growth inhibitory effect that the first component of the amphibody seeks to neutralize is one of the following naturally occuring neurite growth inhibitors: NOGO; myelin associated glycoprotein (MAG); arretin; a proteoglycan; a Sem-receptor; and a member of any one of the families of semaphorins; tenascins, netrins and Eph and ephrins.

In a particular embodiment of the present method, the above mentioned second component of the neuromodulator is a neurotropic molecule, such as (1) a cell adhesion molecule (CAM), e.g. an immunoglobulin superfamily CAM, a cadherin or an integrin, or any functional fragment thereof, or (2) an extracellular matrix molecule (ECM), such as laminin, a netrin or any functional fragment thereof.

In another embodiment, the above mentioned second component is comprised of a neurotrophic molecule selected from the group consisted of (1) neurotrophin family consisting of nerve growth factor (NGF), brain-derived neurotrophic factor (BDNF), neurotropin 3 (NT3) or neurotropin 4 (NT4), (2) a member of the GDNF-subfamily, preferably glial cell derived neurotrophic factor (GDNF) or its relatives neurturin (NTN), persephin (PNP) or artemin (Art), (3) a neuropoietic cytokine, notably ciliary neurotrophic factor (CNTF), leukemia inhibitory factor (LIF) or cardiotrophin-1 (CT-1), any one of the fibroblast growth factors (FGF), hepatocyte growth factor (HGF) or (other factors with documented neuroprotective and stimulatory effects).

The present amphibody may e.g. be prepared by genetic recombination techniques or as a fusion protein.

Thus, a protein engineering method for the production of a cell growth modulator, such as an amphibody, according to the invention may comprise the following general steps:
(a) providing a nucleic acid encoding a suitable first component capable of binding to a target and suppressing, or essentially neutralising, a cell growth inhibitory effect thereof;
(b) providing a nucleic acid encoding a suitable second component capable of stimulating cell growth and/or regeneration;
(c) fusion of said nucleic acids into a recombinant nucleic acid construct;
(d) providing a vector carrying said construct;
(e) insertion of said vector into a suitable host cell; and
(f) expression of the desired regulator.

Accordingly, the present invention also relates to such a nucleic acid encoding the neuromodulator according to the invention as such as well as to any nucleic acid capable of specific hybridization therewith under stringent conditions as defined by standard parameters found e.g. in the patent literature. In case the amphibody is comprised of the two components spaced apart by a linker element, the nucleic acid construct will also comprise an intervening sequence encoding such a linker. Further, another aspect of the invention is a vector comprising a nucleic acid construct according to the invention. Said vector may e.g. be a plasmid, a virus, etc. The host cell may be eucaryotic or procaryotic, such as a bacterial cell, but is preferably a eucaryotic cell, such as an insect cell or any other suitable cell line used for the production of recombinant proteins. General laboratory procedures required in this context can be found in Sambrook et al., Molecular Cloning, A Laboratory Manual, 2^{nd} ed., vol. 1-3, Cold Spring Harbor Laboratory, Cold Spring Harbor, N.Y., 1989. Molecular cloning techniques are found in Berger and Kimmel, Guide to Molecular Cloning Techniques, Methods in Enzymology, vo. 152 Academic Press, Inc., San Diego, Calif.; and Current Protocols in Molecular Biology, F. M. Ausbel et al., eds., Current Protocols, Greene Publishing Associates, Inc. And John Wiley & Sons, Inc., (1994 Supplement). Further, see Freshney, Culture of Animal Cells, A Manual of Basic Technique, 3^{rd} ed., Wiley-Liss, New York, N.Y. (1994) and the references cited therein for a general guide to the culture of cells.

In gene technology, expression systems employing gene fusion constructs have been used widely in the field of protein production, e.g. in order to enhance the production of proteins in eukaryotic and procaryotic cells. For a general review of such techniques, see e.g. Smith and Johnson (1998) Gene 67:31; Hopp et al. (1988) Biotechnology 6:1204; and La Vallie et al. (1993) Biotechnology 11:187. See also U.S. Pat. No. 5,541,087, entitled “Expression and export technology of proteins as immunofusins”. Further, in the examples below, the preparation of an amphibody according to the invention will be disclosed in more detail.

In an alternative embodiment, the invention relates to the a method of producing a nerve cell growth modulator, such as an amphibody, by use of chemical fusion techniques, wherein the desired anti-inhibitory protein is combined with a suitable stimulatory protein. Such a method comprises the steps of
(a) providing a first component capable of binding to a target and suppressing, or essentially neutralising, a nerve cell growth inhibitory effect thereof;
(b) providing a second component capable of stimulating nerve cell growth and/or regeneration;
(c) chemical fusion of said components with a suitable reagent to produce the desired amphibody.
   The components may be proteins, polypeptides, peptides and carbohydrates. Further, in a specific embodiment, in step (c), the ratio between the first and the second component is about 1:1.

More specifically, proteins can be irreversibly linked to each other by a number of chemical procedures. In addition, other molecules than proteins can be chemically linked to proteins (carbohydrates, small organic molecules etc.), making this a very versatile tool to create fusion molecules. Chemical coupling can be performed in a vast number of ways, depending on the molecules to be coupled, the requirements on the final product etc. In general, the coupling can be performed with different degrees of control of the reaction, such that “crude” methods result in an uncontrolled collection of fusion products that can vary in their content of the contributing molecular entities; even large aggregates of fused molecules may be the result of such procedures. In contrast, using procedures where one of the molecular partners is first “activated”, i.e. primed for the subsequent coupling to the other molecular partner, and then let to chemically bind to that latter molecule, a much more predictable result may follow. For example, using bi-functional reagents, with one end reacting with the first molecule, and another end reacting with the second molecule (the procedure very often performed as a two step reaction), results in fused products with the ratio 1:1 of the first and the latter molecule. The mentioned bifunctional reagent may also provide a linker of defined length between the two coupled molecules, which may improve the functional charactersitics of the resulting fusion molecule.

In one specific embodiment, a combination of genetic engineering and chemical coupling is used, such that a protein is designed to carry certain properties/entities allowing a subsequent, chemical coupling to another molecule. An example of this is the engineering of Cysteine or Lysine residues into a recombinant protein, utilizing the SH or epsilon-amine group, respectively, for subsequent coupling.

Purification protocols ensuring that the purified material comprise both the molecular entities as designed, is often used in the production of chemically generated fusion molecules, similar to what is being said for purification of fusion proteins in section 2.1.4.

Thus, both of the above described methods may also comprise a step of purification of the desired modulator from any undesired matter. For methods of protein purification, see e.g. R. Scopes (1982) Protein Purification, Springer-Verlag, N.Y.; Deutcher (1990), Methods in Enzymology vol. 182: Guide to Protein Purification, Academic Press, Inc., N.Y.).

Further references relevant in this context are Clackson T, Hoogenboom H R, Griffiths A D, Winter G. Making antibody fragments using phage display libraries. Nature 1991; 352:624-628; Ford C F, Suominen I, Glatz C E. Fusion tails for the recovery and purification of recombinant proteins. Protein Expression & Purification 1991; 2:95-107; Haber E. Engineered antibodies as pharmacological tools. Immunological Reviews 1992; 130:189-212; Huse W D, Sastry L, Iverson S A, Kang A S, Alting-Mees M, Burton D R, Benkovic S J & Lemer R A. Generation of a large combinatorial library of the immunoglobulin repertoire in phage lambda. Science 1989; 246:1275-81; Morrison, S L, in Borrebaeck, C A K (ed.): Antibody Engineering, 1995; Murphy J R, Bishai W, Williams D, Bacha P, Borowski M, Parker K, Boyd J, Waters C, Strom T B. Genetic assembly and selective toxicity of diphtheria-toxin-related polypeptide hormone fusion proteins. Biochemical Society Symposia 1987; 53:9-23; Murray K M, Dahl S L. Recombinant human tumor necrosis factor receptor (p75) Fc fusion protein (TNFR:Fc) in rheumatoid arthritis. Annals of Pharmacotherapy 1997; 31:1335-1338; Rader C, Barbas C F 3rd. Phage display of combinatorial antibody libraries. Current Opinion in Biotechnology 8:503-8, 1997; Sambrook J, Fritsch E F, Maniatis T. Molecular Cloning. A Laboratory Manual. 2nd ed. Cold Spring Harbor Laboratory Press, Cold Spring Harbor, 1989; Samuelsson A, Yari F, Hinkula J, Ersoy O, Norrby E, Persson M A A. Human antibodies from phage libraries: Neutralizing activity against human immunodeficiency virus type 1 equally improved after expression as Fab and IgG in mammalian cells. Eur J Immunol 1996; 26:3029-3034; and Uhlen M, Forsberg G, Moks T, Hartmanis M, Nilsson B. Fusion proteins in biotechnology. Current Opinion in Biotechnology 1992; 3:363-369.

A third aspect of the present invention is a cell, which expresses a neuromodulator according to the invention. Such a cell may be a eucaryotic or procaryotic cell, preferably a human cell, such as a fibroblast, a glial cell, such as an oligodendroglial cell or Schwann cell or olfactory ensheathing glial cell.

### Parmaceutical Applications

A further aspect of the present invention is a neuromodulator according to the invention for use as a medicament. Thus, the invention also relates to the use of the present neuromodulator in the manufacture of a medicament for the treatment and/or prevention of e.g. spinal cord injury, brain trauma, stroke, retinal and optic nerve lesions, neurodegenerative diseases, neuromuscular diseases, autoimmune diseases of the nervous system, tumors of the central nervous system etc. Consequently, the invention also includes a pharmaceutical preparation prepared according to said use. The invention also encompasses the above defined uses, wherein a cocktail, i.e., any mixture, of different neuromodulators according to the invention is used.

Thus, the present invention also relates to methods of treatment and/or prevention of the above defined conditions, wherein an amphibody or an amphibody cocktail is administered to a patient in need of therapy.

In one embodiment, the preparation may be in a form suitable for intravenous administration. As has been demonstrated repeatedly in the literature, there are several ways in which proteins can be effectively delivered into brain and spinal cord, all of which are useful in the present context. The following are examples of possible delivery strategies:

Intravenous administration: The intact central nervous system has a blood-brain barrier that severely limits entry of proteins into the brain or spinal cord if delivered into the blood stream. However, in trauma and certain other diseases and disturbances of the brain or spinal cord, the blood-brain barrier itself is damaged. Consequently, in such conditions, intravenous delivery of appropriate doses of amphibodies will lead to therapeutically effective levels of amphibodies specifically in those injured areas of the CNS having barrier damage.

Intraparenchymal, intraventricular or intrathecal administration: In regard of cells that make and secrete amphibodies, intraparenchymal, intraventricular or intrathecal administration may be used. Well-documented techniques exist to transfect cells with gene constructs leading to high production and secretion of the corresponding proteins, see below in the context of gene and cell therapy metods. Cell suspensions can be injected using microneedles and deposited with stereotaxic precision in desired areas of the central nervous system.

Direct gene delivery: Several well-documented techniques also exist for direct gene delivery to the central nervous system. Thus, the amphibody genes constructed according to the present invention can be carried by viral vectors or by other means such as liposomes etc. using stereotaxic injection techniques. Cells in the neighborhood will take up the new gene and produce amphibodies locally. In animal experiments different forms of neurotrophic viruses, e.g. herpes virus, modified so it cannot replicate, have been successfully used for other proteins. At present the best choice for human applications appears to be the adeno-associated virus which is carried by a large proportion of the human population and has been declared a nonpathogen. Non-replicating adenovirus is another option.

Oral administration: Oral administration may lead to degradation in the gastrointestinal canal, and, accordingly, such methods must be especially adapted to avoid enzyme-catalyzed degradation and to maintain effective concentrations.

For a brief review of general methods of drug delivery, see e.g. Langer, Science 249:527-1533 (1990).

In addition, the present invention also relates to a cell which produces one or more amphibodies according to the invention for use as a medicament per se. Thus, the invention also encompasses a pharmaceutical preparation, which comprises a cell producing one or more amphibodies according to the invention together with a pharmaceutically acceptable carrier for use in gene or cell therapy methods. Accordingly, the present invention also encompasses methods of treatment and/or prevention of disease and/or trauma, wherein a cell producing an amphibody, or several different cells producing various amphibodies, according to the invention, is administered to a patient in need of therapy. Thus, the present cells producing amphibodies may be used in treatment schemes aimed at e.g. spinal cord injury, brain trauma, stroke, retinal and optic nerve lesions, neurodegenerative diseases, neuromuscular diseases, autoimmune diseases of the nervous system, tumors of the central nervous system etc. An especially advantageous method for treatment according to the invention is a method, wherein amphibody producing cells are administered, which cells are obtained from the individual receiving the therapy. The cells may for example be fibroblasts originating from the present patient. Thus, using this method, immunological rejection of non-autologous biological material is circumvented.

For a review of general methods of gene therapy, see e.g. Anderson, Science (1992) 256:808-813; Nabel and Felgner (1993) TIBTECH 11:211-217; Mitani and Caskey (1993) TIBTECH 11:162-166; Mulligan (1993) Science 926-932; Dillon (1993) TIBTECH 11: 167-175; Miller (1992) Nature 357:455-460; Van Brunt (1988) Biotechnology 6(10):1149-1154; Vigne (1995) Restorative Neurology and Neuroscience 8:35-36; Kremer and Perricaudet (1995) British Medical Bulletin 51(1) 31-44; Haddada et al. (1995) in Current Topics in Microbiology and Immunology Doerfler and Böhm (eds) Springer-Verlag, Heidelberg, Germany; and Yu et al., Gene Therapy (1994) 1:13-26. Further, cell therapy as a strategy is e.g. described in Nature, vol 392, supp, 30 Apr. 1998, by Fred H. Gage.

### Uses of Amphibodies

The basic amphibody concept consists of a molecule having one end that binds to and neutralizes a neurite growth inhibitory site and another end carrying a neurite growth-stimulatory molecule. By combining such anti- and pro- candidates, such as the ones exemplified herein, a multitude of amphibodies can be produced with multifarious properties for specific purposes. Different clinical settings would require their appropriate set of amphibodies, that would consist of anti- and pro-components that are known to be active in the particular setting. For example, different amphibodies could be used in peripheral nerve injuries, optic nerve injuries and spinal cord injuries, where the local milieu is not the same. Also, different amphibodies could be beneficial for different types of axons, e.g. motor or sensory, since it is known that different neurons respond to a specific agenda of trophic factors, as is decided by the receptors they are expressing and the maturation stage they are in, and given the alterations that an axotomy or other lesions would confer upon the neuron and its axon. Furthermore, considering the complexity of the nervous tissue and the variety of regenerating neurons, amphibodies can be used in the following ways:

a) Complex amphibodies are constructed that neutralize a single inhibitory target but express more than one neurotrophic or neurotropic properties at the other end.

b) Combinations of amphibodies and complex amphibodies with variable anti-inhibitory and stimulatory ends are used simultaneously, administered as a “cocktail”, to achieve maximum benefit for regeneration.

Thus, for example, by combining an antibody against NOGO with various neurotrophic factors, the following amphibodies may be obtained: anti-NOGO/NGF; anti-NOGO/BDNF; anti-NOGO/NT3; anti-NOGO/NT4; anti-NOGO/GDNF; anti-NOGO/NTN; anti-NOGO/CNTF; anti-NOGO/CT-1; anti-NOGO/bFGF; anti-NOGO/L1-CAM; etc.

In a clinical scenario, all these could be administered as a mixture or an “amphibody cocktail” for example in spinal cord injury, because it is known that in spinal cord injury, NOGO is a major myelin-derived inhibitor of axonal growth, while all the pro-end components have documented trophic or tropic effects on regenerating axons in an experimental spinal cord injury setting. Thus the axons, instead of the NOGO inhibitor, will be faced with multigenerous trophic signals that will induce and amplify the regenerating process. Adding to the above, administration of a second or third “battery of amphibodies” with different anti-ends, e.g. anti-MAG, anti-neurocan etc., will knock out more axonal outgrowth inhibitors and suppressors while enriching the field with more positive signals, converting the initially nonpermissive or hostile axonal environment into an increasingly trophic one. Protocols of specific amphibody cocktails will be tailored for different neuronal diseases in a way similar to the chemotherapeutic protocols used to treat different malignancies.

Further, amphibodies will be useful research tools in animal experiments, in vitro and in vivo.

### ADVANTAGES OF THE PRESENT INVENTION

According to the prior art, it is known that transected nerve fibers in the brain and spinal cord can regenerate given the appropriate environment. This does not occur normally, but has been repeatedly demonstrated in the laboratory, for instance by presenting such nerve fibers with the permissive and trophic environment of a peripheral nerve, in which case cut nerve fibers of CNS origin readily regenerate inside the peripheral nerve. The amphibodies according to the present invention for the first time enables to achieve this vital turnabout change of milieu by on the spot neutralizing negative cues and in situ exposing positive modulators of axonal growth in a “sugarcoat” fashion.

Further, it has been demonstrated in the literature that neutralisation of inhibitory proteins on the surface of oligodendroglial cells can in itself elicit a degree of axonal regeneration, albeit limited, since this approach turns a negative environment into mere neutral. However, there are many more other inhibitory factors in the nervous system. The present amphibodies will be more powerful agents, since not only will they nullify a multitude of inhibitory factors, but they will simultaneously supplement axonotrophic/tropic elements in the region, precisely on the spot of the previously exposed inhibitory sites, turning a negative environment into a positive one rather than a neutral one. This is the essence of molecular guising, which clearly distinguishes the function of the present neuromodulators from the prior art.

The pioneer axon will not run the risk of being brought to standstill, since elongation takes place at the tip of the lamellipodium/filopodium by constitutive membrane traffic. The leading filopodia act as sensors by exploring the terrain and will direct themselves towards the next “attractive choice points”, presented by the pro-ends of docked/stationed amphibodies in the immediate proximity. Thus, the amphibodies according to the invention will both support and sustain the process of axonal advance and regeneration by introducing the tip onto a “chemoattractant railway”.

Conclusively, the fact that CNS axons have an intrinsic capability to regenerate together with the documented actions of the disinhibiting and the stimulating components of amphibodies, as well as the feasibility of their delivery discussed herein, make them ideal one-molecule tools for reparative treatments by controllably and safely modifying the local environment to the benefit of regenerating fibers, in a regionspecific and chemospecific manner, without the risks entailed by use of the prior art simple mixtures of components.

In this context, see e.g. Schwab M. E.: Molecules inhibiting neurite growth: A minireview. in Neurochemical Research. 21(7):755-61, 1996 July; Spillmann A A. Amberger V R. Schwab M E.: High molecular weight protein of human central nervous system myelin inhibits neurite outgrowth: An effect which can be neutralized by the monoclonal antibody IN-1 in European Journal of Neuroscience, 9(3):549-55, 1997 March; Schwab M E. Kapfhammer J P. Bandtlow C E.: Inhibitors of neurite growth. in Annual Review of Neuroscience, 16:565-95, 1993; Fitch M T. Silver J.: Glial cell extracellular matrix: Boundaries for axon growth in development and regeneration, in Cell & Tissue Research, 290(2):379-84, 1997 November; and Hoke A. Silver J.: Proteoglycans and other repulsive molecules in glial boundaries during development and regeneration of the nervous system, in Progress in Brain Research, 108:149-63, 1996.

### DETAILED DESCRIPTION OF THE DRAWINGS

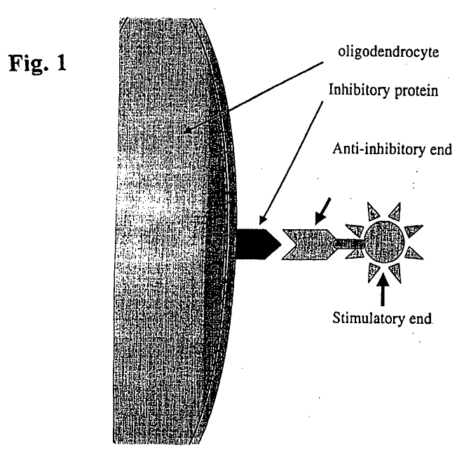

FIG. 1 shows the amphibody principle. The amphibody according to the invention consists of two parts. In this example the anti-end binds to, and neutralizes an inhibitory site. This dictates the in situ localization of the pro-end which is thus exposed to regenerating neurites.

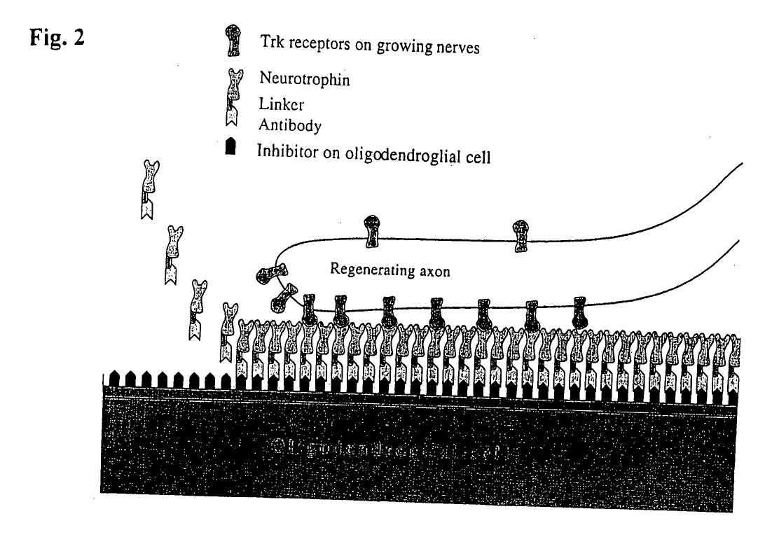

FIG. 2 is a schematic illustration of the function of amphibodies according to the invention. In this case the anti-ends bind to and cap the neurite inhibitory sites expressed on the surface of oligodendroglial cells. The pro-end in this example is a neurotrophin. The growing neurite carries neurotrophin receptors of the Trk class. The amphibody pro-ends become aligned to form a chemoattractant “railway” along which the neurite will regenerate.

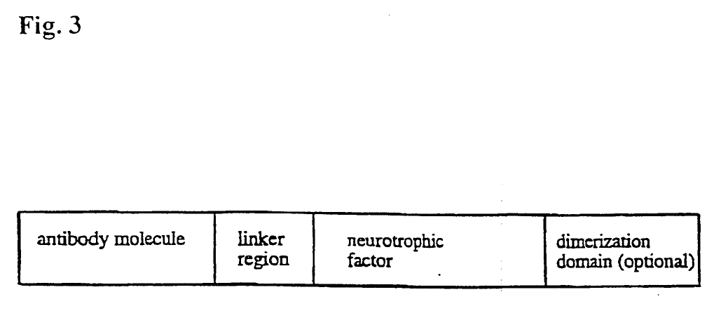

FIG. 3 shows the design and construction of an amphibody according to the invention as three major parts: the antibody, the neurotrophic factor, and a linker in between the two former ones.

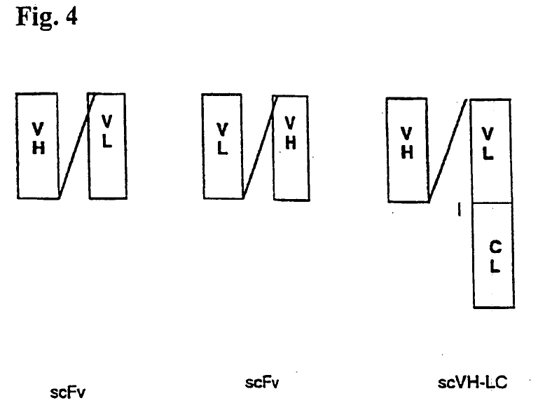

FIG. 4 shows how single chain constructs may include both the variable domains (VH and VL) linked via a peptide linker to form a continuous antigen binding molecule, a so called scFv. The order of the genetic elements may be either VH-linker-VL, or vice versa: VL-linker-VH. For certain purposes, one being stability of the molecule, the constant domain of the light chain may be included, giving a whole Ig light chain linked to the VH region (so called scVH-LC format).

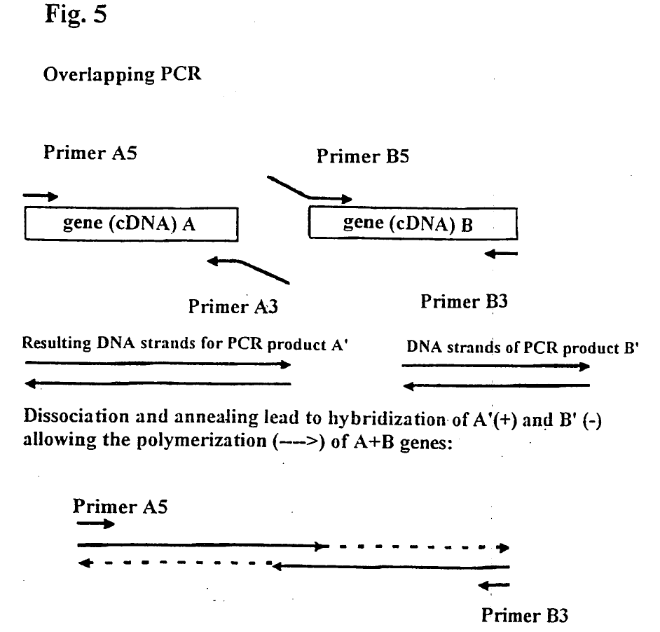

FIG. 5 shows the principles of overlapping PCR in the construction of the genes coding for the amphibodies according to the invention. In this technique, the different portions of the gene (e.g. A and B) to be assembled will be amplified by PCR using primers that include sequence motifs allowing the hybridization of PCR products that should be assembled adjacent to each other. Thus, PCR products A′ and B′ are obtained in a first step. In a second PCR reaction, the DNA strands of the two PCR products A′ and B′ to be assembled in the arrangement A-B are dissociated, and when annealed, a proportion of the strands will hybridize in a cross fashion: sense strand of A′ with anti-sense strand of B′ and v.v. In the polymerization step, the extended product A+B will be produced, facilitated by the addition of primers hybridising to the 5′ end of A and to the 3′ end of B.

### EXPERIMENTAL

Below, listings of suitable components of the present amphibodies are presented, followed by preparation methods thereof and examples. It is to be understood that the herein presented examples in no way are intended to limit the scope of the invention as defined by the appended claims. All references given below and elsewhere in the present specification are included herein by reference. For clarity, this part of the present specification has been divided into separate headlines as presented in the table of contents presented below:
1. AMPHIBODY COMPONENTS
1.1 First component: Inhibitors/suppressors of:
(i) Nogo
(ii) MAG
(iii) Proteoglycans
(iv) Semaphorin family
(v) Neutralization of Sem-receptors
(vi) Tenascins
(vii) Netrins
(viii) Eph and ephrins
1.2 Second component
1.2.1 Neuroiropic molecules
(a) Cell Adhesion Molecules
   (i) Immunoglobin superfamily
   (ii) Cadherin superfamily
   (iii) Integrin superfamily
(b) Extracellular Matrix Molecules
1.2.2 Neurotrophic molecules
(i) Neurotrophins (NGF family)
(ii) GDNF subfamily
(iii) Neurokines
(iv) Fibroblast growth factors (FGF family)
(v) Hepatocyte growth factor (HGF)/scatter factor
(vi) Other factors
2. CONSTRUCTION OF AMPHIBODIES
2.1 General methods
2.1.1 Antibodies; introduction
2.1.2 Hybridoma methods
2.1.3 Genetic methods
2.1.4 Fusion proteins
2.2 Putative design of amphibodies
2.2.1 The antibody portion
2.2.2 The linker region
2.2.3 The neurotrophic portion
2.2.4 Dimerization domain
2.3 Chemical methods
2.4 Example 1

### 1. Amphibody Components

### 1.1 First Component: Inhibitors/Suppressors of:

(i) Nogo (previously NI-35/250)

The existence of CNS white matter local factors that confer nonpermissiveness to axon regrowth has been hinted before the actual discovery by Martin Schwab and colleagues of myelin-associated neurite-outgrowth inhibitors in various mammals and their negative role in CNS regeneration in vitro and in vivo. The possible therapeutic potential of a monoclonal neutralizing antibody, IN-1, has been established by in vitro and in vivo studies of CNS lesions, notably optic nerve lesions and experimental spinal cord injury. An additional action of neurite inhibitors in restricting plasticity in CNS and again a beneficial effect of IN-1 have also been shown. Genetic engineering has allowed the production of the Fab fragment of the IgM IN-1 antibody, which has proven to have neutralizing properties. (See M E van der Haar, M S Chen A B Huber M E Schwab: Myelin-associated outgrowth inhibitory factors: recombinant expression of Nogo (NI-220/250) 616.7 in Soc. Neurosci. Abstr., Vol. 24 Part 2, p. 1559, 1998; Developmental expression pattern and functional analysis of Nogo (formerly NI-35/250), a major inhibitor of CNS regeneration 616.6 in Soc. Neurosci. Abstr., Vol. 24 Part 2, p. 1559,1998; Bandtlow C. Schiweck W. Tai H H. Schwab M E. Skerra A.: The Escherichia coli-derived Fab fragment of the IgM/kappa antibody IN-1 recognizes and neutralizes myelin-associated inhibitors of neurite growth, in European Journal of Biochemistry. 241(2):468-75, 1996 Oct. 15; and Spillmann A A, et al. Identification and characterization of a bovine neurite growth inhibitor (bNI-220). in J Biol Chem. 1998 Jul. 24; 273(30): 19283-93.)

### (ii) MAG (Myelin Associated Glycoprotein)

MAG is a neural adhesion molecule of the Immunoglobulin superfamily, and differs from the other Ig superfamily CAMs in that it is the only one to lack FNIII repeats. Expressed by glial cells it has been ascribed a role in central and peripheral axon myelination by mediating and modulating glial-axon contact. Accordingly it seems to be involved in the regenerating process following myelin breach in both the PNS and the CNS. While MAG's role is not as clear-cut as that of NOGO, there are enough recent studies advocating MAG-related inhibitory action on axonal regeneration, especially in CNS lesions. It is even reported to exist in vivo in a soluble form, similarly inhibitory.

In this context, see e.g. Tang S. Woodhall R W. Shen Y J. De Bellard M E. Saffell J L. Doherty P. Walsh F S. Filbin M T.: Soluble myelin-associated glycoprotein (MAG) found in vivo inhibits axonal regeneration, in Molecular & Cellular Neurosciences, 9(5-6):333-46, 1997; Shen Y J. DeBellard M E. Salzer J L. Roder J. Filbin M T.: Myelin-associated glycoprotein in myelin and expressed by Schwann cells inhibits axonal regeneration and branching, in Molecular & Cellular Neurosciences, 12(1-2):79-91, 1998 September; Tang S. Woodhall R W. Shen Y J. deBellard M E. Saffell J L. Doherty P. Walsh F S. Filbin M T.: Soluble myelin-associated glycoprotein (MAG) found in vivo inhibits axonal regeneration, in Molecular & Cellular Neurosciences, 9(5-6):333-46, 1997; Li M. Shibata A. Li C. Braun P E. McKerracher L. Roder J. Kater S B. David S.: Myelin-associated glycoprotein inhibits neurite/axon growth and causes growth cone collapse, in Journal of Neuroscience Research, 46(4):404-14, 1996 Nov. 15; and Lai C. Watson J B. Bloom F E. Sutcliffe J G. Milner R J.: Neural protein 1B236/myelin-associated glycoprotein (MAG) defines a subgroup of the immunoglobulin superfamily, in Immunological Reviews, 100:129-51, 1987 December.

(iii) Proteoglycans (PG)

The chondroitin and keratan sulfate proteoglycans have been implicated as both positive and negative modulators of axonal growth in the developing nervous system and after injury when most of them are upregulated and have inhibitory properties, e.g. neurocan, phosphocan, versican, biglycan, decorin, NG2, DSD-1. As an example, NG2 has been shown in vitro to provide an unfavorable substrate for axonal growth, may in vivo define nonpermissive areas for axonal extension and its increased postinjury expression may contribute to the failure of damaged CNS axons to regenerate successfully. As another example, neurocan binds to L1 and NCAM and inhibits neuronal adhesion and neurite outgrowth. Injury-induced PG were shown to inhibit the potential for laminin-mediated axon growth on astrocytic scars.

In this context, see e.g. Ruoslahti E (1996) Brain ECM, Glycobiology 6:489-492; Nishiyama A Dahlin K J (1991): The primary structure of NG2, a novel membrane-spanning proteoglycan in J Cell Biol 114:359-371; Höke A Silver J (1996): Proteoglycans and other repulsive molecules in glial boundaries during development and regeneration of the nervous system in Prog Brain Res 108:149-163; Friedlander D (1994): The neuronal chondroitin sulphate PG neurocan binds to NCAMs Ng-CAM/L1/NILE and N-CAM and inhibits neuronal adhesion and neurite outgrowth, in J Cell Biol 125:669-680; McKeon R J. Hoke A. Silver J.: Injury-induced proteoglycans inhibit the potential for laminin-mediated axon growth on astrocytic scars, in Experimental Neurology, 136(1):32-43, 1995 November; Dou C-L, Levine J M (1994): Inhibition of neurite growth by the NG2 chondroitin sulfate proteoglycan in J neurosc 14:7616-7628.

### (iv) Semaphorin Family Members

A large family of secreted and transmembrane proteins. They fall into seven subclasses. Some members function as axon repellents during development. Neuronal populations respond differentially to some of these guidance molecules. Neurotrophins can modulate growth cone response to certain semaphorins, e.g. collapsin-1. Also collapsin-1 exhibits the common theme of dimerization as a prerequisite for growth cone collapsing activity. Vertebrate collapsin 1 is a potent inducer of sensory growth cone collapse and has been implicated as a diffusible chemorepellent that patterns sensory axon projections in the spinal cord.
Semaphorins: Main action, chemorepulsive.
Collapsin: Main action: Outgrowth-suppressing.

In this context, see e.g. Wright D E. White F A. Gerfen R W. Silos-Santiago I. Snider W D.: The guidance molecule semaphorin III is expressed in regions of spinal cord and periphery avoided by growing sensory axons, in Journal of Comparative Neurology, 361(2):321-33, 1995 Oct. 16; Varela-Echavarria A. Tucker A. Puschel A W. Guthrie S.: Motor axon subpopulations respond differentially to the chemorepellents netrin-1 and semaphorin D, in Neuron. 18(2):193-207, 1997 February; and Giger R J. Pasterkamp R J. Heijnen S. Holtmaat A J. Verhaagen J.: Anatomical distribution of the chemorepellent semaphorin III/collapsin-I in the adult rat and human brain: Predominant expression in structures of the olfactory-hippocampal pathway and the motor system, in Journal of Neuroscience Research, 52(1):27-42, 1998 Apr. 1.

### (v) Neutralization of Sem-Receptors

One could indirectly achieve the effect of modulating semaphorin actions by interfering with their bindings on semaphorin receptors, e.g. neuropilin and plexin semaphorin/receptor families. Neuropilin 1 interacting with its semaphorin ligands was shown to play a crucial role in peripheral nerve projection in mice. Neuropilin mediates nerve fiber fasciculation. Plexins contain sema domains and can function as homophilic cell adhesion molecules. A plexin family semaphorin receptor, VESPR (viral-encoded semaphorin protein receptor), is expressed in a variety of hæmatopoietic cells mediating inflammatory reactions.

In this context, see e.g. He Z. Tessier-Lavigne M.: Neuropilin is a receptor for the axonal chemorepellent Semaphorin III, in Cell 90(4):739-51, 1997 Aug. 22.

### (vi) Tenascins (TN)

Tenacsins is a family of extracellular matrix glycoproteins of glial and neuronal cells with effects on neuronal cell adhesion, growth cone guidance and neuronal polarity. They have diverse functions during neural development, e.g. tenascin-R (restrictin) binds IgCAM contactin/F11 and enhances F11-mediated neurite outgrowth, and in perturbation studies antibodies against tenascin delayed endplate reinnervation. Tenascin-R and tenascin-C are also expressed in the lesioned nervous tissue and have both stimulatory and anti-adhesive/inhibitory properties for axon growth. For their bimodal potential TN are listed as pro-end/targets as well. As a component of the glial scar following CNS injury it is supposed to have an inhibitory role by reducing neurite outgrowth.

In this context, see e.g. Meiners S. Geller HM.: Long and short splice variants of human tenascin differentially regulate neurite outgrowth, in Molecular & Cellular Neurosciences, 10(1-2):100-16, 1997; Faissner A.: The tenascin gene family in axon growth and guidance, in Cell & Tissue Research, 290(2):331-41, 1997 November; Dorries U. Taylor J. Xiao Z. Lochter A. Montag D. Schachner M.: Distinct effects of recombinant tenascin-C domains on neuronal cell adhesion, growth cone guidance, and neuronal polarity, in Journal of Neuroscience Research, 43(4):420-38, 1996 Feb. 15; and Norenberg U. Hubert M. Rathjen F G.: Structural and functional characterization of tenascin-R (restrictin), an extracellular matrix glycoprotein of glial cells and neurons, in International Journal of Developmental Neuroscience 14(3):217-31, 1996 June.

(viii) Netrins

They are a small family of bifunctional guidance cues mainly involved in neural development, capable of attracting some axons and repelling others. They are diffusible and related to the larger laminins, which are themselves ECM molecules with effects on axon guidance.

In this context, see e.g. Culotti J G. Kolodkin A L.: Functions of netrins and semaphorins in axon guidance, in Current Opinion in Neurobiology 6(1):81-8, 1996 February.

(viii) Eph and Ephrins

The Eph family of receptors represents the largest family of receptor tyrosine kinases with 14 members at present, subdivided into two classes (EphA and EphB subclasses), because of their binding specificities to two classes of Eph ligands, ephrinsA and ephrinsB, altogether. It has been reported that both Eph and ephrins can function both as receptors and as ligands, which is why both are included here. They are known to be short-range repulsive guidance cues, involved in the establishment of many topographic projections, axon fasciculation and even neural precursor cell migration.

In this context, see e.g. Flanagan J G. Vanderhaeghen P.: The ephrins and Eph receptors in neural development in Annual Review of Neuroscience; Gale N W. Yancopoulos G D.: Ephrins and their receptors: a repulsive topic? in Cell & Tissue Research. 290(2):227-41, 1997 November; and Unified nomenclature for Eph family receptors and their ligands, the ephrins. Eph Nomenclature Committee [letter]. Cell. 90(3):403-4, 1997 Aug. 8.

### 1.2 Second Component

### 1.2.1 Neurotropic Molecules

### (a) Cell Adhesion Molecules (CAMs)

### (i) Immunoglobulin Superfamily; (Ca-Independent)

The Ig superfamily is one of the largest group of proteins expressed on extending axons and glial cells and mediate contact-dependent axonal growth, pathfinding and even fasciculation and myelination, from nematodes to humans. They are cell surface glycoproteins that serve as ligands to support cell adhesion, neurite outgrowth and guidance by homophilic binding and by complex heterophilic binding to other CAMs, integrins and ECM proteins. Ig CAMs are widely expressed during development and are very important for correct neurohistogenesis as is exemplified in human neurological disorders caused by L1-CAM mutations, e.g. X-linked hydrocephalus and X-linked spastic paraplegia, the L1-CAM gene being located on the human X chromosome. Also mice lacking the L1 gene show erroneous corticospinal tract guidance pattern. The Ig CAMs are transmembrane (NCAM, L1-CAM) or membrane-bound (TAG-1/axonin-1, which is GPI-anchored) and they are typified by having an extracellular portion composed of a few Ig domains situated distally (at their N-terminal) and tandem fibronectin type III (FNIII) repeats found proximally (closer to the membrane). It is postulated that their homophilic binding involves the terminal Ig domains when two CAMs interact pair-wise in an antiparallel orientation. Their modular domain organization and the fact that their function can sometimes be pinpointed on some or single Ig-domains, make them suitable for the protein engineering technology used to create amphibodies. For example, in the human L1 -CAM its activity has been attributed to the second Ig domain and L1-CAM is still fully active even when lacking its endogenous cytoplasmic domain, which means that as a component of the amphibody pro-moiety it will still mediate cell adhesion and neurite growth. In vitro L1 is a robust axonal growth substrate, whereon RGC and DRG growth cones take the form of large lamellipodia with short filopodia (opposite to what is seen with laminin as is discussed below). When retinal ganglion cells (RGC) are cultured on purified protein substrates, their growth cones are twice as adhesive to L1 as to laminin, probably because of the longer lamellipodium. Amphibodies with an L1 pro-moiety introduce an axon growth promoting property to the environment of the regenerating axon by homophilic binding with the abundant L1 on the axonal membrane, while the anti-moiety of the amphibody at the same time would neutralize a chosen inhibitory cue, e.g. Nogo and also dock the amphibody on a surface, thereby creating an “adhesive railway” for the extending axon. Another amphibody pro-moiety is the TAX-1 gene product, the human equivalent of the mouse TAG-1/axonin-1, another Ig CAM axon-associated adhesion molecule that can induce neurite growth by homophilic interaction or by heterophilic interaction with L1, and has demonstrated its potential in vitro and in vivo.

NCAM (the prototype molecule of the Ig CAM family)

L1-subfamily (L1-CAM/5G3, neurofascin, NrCAM)

TAX-1 gene product (human equivalent of rat TAG-1/axonin-1)

In this context, see e.g. Brümmendorf T, Rathjen F G (1995): Cell adhesion molecules 1:Ig superfamily in Protein profile 2:963-1108; Schachner M.: Families of neural adhesion molecules in Ciba Foundation Symposium 145:156-69, discussion 169-72, 1989; Walsh F S. Doherty P.: Neural cell adhesion molecules of the immunoglobulin superfamily: Role in axon growth and guidance in Annual Review of Cell & Developmental Biology 13:425-56, 1997; Chothia C., Yvonne Jones E: The molecular structure of cell adhesion molecules, in Annu. Rev. Biochem. 1997. 66:823-62; Martini R.: Expression and functional roles of neural cell surface molecules and extracellular matrix components during development and regeneration of peripheral nerves, in Journal of Neurocytology. 23(1):1-28, 1994 January; Grumet M. Nr-CAM: A cell adhesion molecule with ligand and receptor functions, in Cell & Tissue Research 290(2):423-8, 1997 November; Rathjen F G. Norenberg U. Volkmer H.;: Glycoproteins implicated in neural cell adhesion and axonal growth, in Biochemical Society Transactions 20(2):405-9, 1992 May; Brummendorf T. Rathjen F G.: Structure/function relationships of axon-associated adhesion receptors of the immunoglobulin superfamily in Current Opinion in Neurobiology 6(5):584-93, 1996 October.

Hortsch M.: The L1 family of neural cell adhesion molecules: Old proteins performing new tricks, in Neuron 17(4):587-93, 1996 October; Kamiguchi H. Lemmon V.: Neural cell adhesion molecule L1: Signaling pathways and growth cone motility in Journal of Neuroscience Research. 49(1):1-8, 1997 Jul. 1; Lemmon V. Farr K L. Lagenaur C.: L1-mediated axon outgrowth occurs via a homophilic binding mechanism, in Neuron 2(6):1597-603, 1989 June; Lagenaur C. Lemmon V.: An L1-like molecule, the 8D9 antigen, is a potent substrate for neurite extension, in Proceedings of the National Academy of Sciences of the United States of America 84(21):7753-7, 1987 November.

Ruegg M A. Stoeckli E T. Lanz R B. Streit P. Sonderegger P.: A homologue of the axonally secreted protein axonin-i is an integral membrane protein of nerve fiber tracts involved in neuritefasciculation, in Journal of Cell Biology 109(5):2363-78, 1989 November; Harel R. Futerman A H.: A newly-synthesized GPI-anchored protein, TAG-1/axonin-1, is inserted into axonal membranes along the entire length of the axon and not exclusively at the growth cone, in Brain Research 712(2):345-8, 1996 Mar. 18; Kozlov S V. Giger R J. Hasler T. Korvatska E. Schorderet D F. Sonderegger P.: The human TAX1 gene encoding the axon-associated cell adhesion molecule TAG-1/axonin-1: Genomic structure and basic promoter, in Genomics 30(2):141-8, 1995 Nov. 20; and Felsenfeld D P. Hynes M A. Skoler K M. Furley A J. Jessell TM.: TAG-1 can mediate homophilic binding, but neurite outgrowth on TAG-1 requires an L1-like molecule and beta 1 integrins, in Neuron 12(3):675-90, 1994 March.

### (ii) Cadherin Superfamily CAMs (Ca-Dependent)

Cadherins are morphoregulatory cell adhesion molecules that mediate cell-cell adhesion in a calcium-dependent and mostly homophilic manner. Many cadherin subtypes are known to be expresed in the developing and mature CNS, each with a restricted pattern of expression and a specific function in neural circuit formation. They are involved in neurite outgrowth, navigation and fasciculation, target recognition, synaptogenesis and synaptic plasticity. Their expression is most prominent during active neurite outgrowth. As an example, N-cadherin has been localized to the growth cone of sensory neurons and purified N-cadherin is a potent substrate for the rapid induction and elongation of neurites from retinal, sensory ganglionic and other neurons in vitro. In vitro N-cadherin-positive sensory axons grow preferentially on N-cadherin expressing cells, which illustrates the importance of cadherins for axonal navigation.

In this context, see e.g. Cadherins and tissue formation: Integrating adhesion and signaling, Kris Vleminckx, Rolf Kemler BioEssays. Volume 21, Issue 3, 1999 211-220 ; Tomaselli K J. Neugebauer K M. Bixby J L. Lilien J. Reichardt L F.: N-cadherin and integrins: two receptor systems that mediate neuronal process outgrowth on astrocyte surfaces, in Neuron 1(1):3343, 1988 March.

(iii) Integrin Superfamily

Integrins form a family of heterodimeric cell surface receptors whose name reflects the role of these molecules to integrate the intracellular skeleton with the extracellular matrix. They exist on all cells. They are made up from the combination of 16 α and 8 β subunits that combine to yield 20 unique heterodimers, exist in activated and nonactivated forms, and mediate innumerable cell-cell, cell-matrix interactions. In the nervous system, they are believed to be implicated in neuronal migration, axon fasciculation and morphogenesis, and play a central role in axon outgrowth and neurite elongation in development and regeneration by interacting with CAM, e.g. L1 or extracellular matrix (ECM) proteins, e.g. laminin, fibronectin, and collagen IV.

In this context, see e.g. Reichardt L F. Bixby J L. Hall D E. Ignatius M J. Neugebauer K M. Tomaselli K J.: Integrins and cell adhesion molecules: neuronal receptors that regulate axon growth on extracellular matrices and cell surfaces, in Developmental Neuroscience 11 (4-5):332-47, 1989; McKerracher L. Chamoux M. Arregui C O.: Role of laminin and integrin interactions in growth cone guidance in Molecular Neurobiology 12(2):95-116, 1996 April; Reichardt L F. Bixby J L. Hall D E. Ignatius M J. Neugebauer K M. Tomaselli K J.: Integrins and cell adhesion molecules: neuronal receptors that regulate axon growth on extracellular matrices and cell surfaces, in Developmental Neuroscience 11(4-5):332-47, 1989; Smith J W. Hu D. Satterthwait A. Pinz-Sweeney S. Barbas C F 3rd: Building synthetic antibodies as adhesive ligands for integrins, in Journal of Biological Chemistry 269(52):32788-95, 1994 Dec. 30; and Tomaselli K J. Neugebauer K M. Bixby J L. Lilien J. Reichardt L F.: N-cadherin and integrins: Two receptor systems that mediate neuronal process outgrowth on astrocyte surfaces, in Neuron. 1(1):33-43, 1988 March.

(b) Extracellular Matrix (ECM) molecules/macromolecules

ECM molecules are produced by nonneuronal cells such as glial cells and fibroblasts and serve as templates for axonal growth and elongation. Axons interact with them by means of integrins and CAMs. The molecule with the most pronounced role as an axon-supporter is laminin produced by astrocytes in development and after injury. When retinal ganglion cells or dorsal root ganglion cells are plated on laminin their growth cones take the form of short lamellipodial expanses with long filopodial processes, which is in contrast to how they grow on L1, though they grow equally well on both laminin and L1. Laminin has been reported to be able to override the inhibitory effects of PNS and CNS myelin-derived inhibitors. Another astrocytic-derived ECM protein is vitronectin. Vitronectin has been shown to promote retinal neurite outgrowth. The diffusible tenascins, although acting in a bimodal fashion, have been in some cases shown to have positive effect on axonal growth. The repetitive structure of some ECM molecules is an advantage to the manufacture of amphibodies. Netrins and tenascins with neurite-growth promotory actions can be included here.

In this context, see e.g. McKerracher L. Chamoux M. Arregui CO.: Role of laminin and integrin interactions in growth cone guidance, in Molecular Neurobiology 2(2):95-116, 1996 April; Letourneau P C. Condic M L. Snow D M.: Interactions of developing neurons with the extracellular matrix, in Journal of Neuroscience 14(3 Pt 1):915-28, 1994 March; Luckenbill-Edds L (1997): Laminin and mechanism of neuronal outgrowth in Brain Res Rev 232:1-27.

### 1.2.2 Neurotrophic Molecules

### (i) Neurotrophins (NGF Family)

The neurotrophins are potent neuronal regulators with broad range of bioactivities in the nervous system. Numerous in vitro and in vivo studies in different models of nervous system injury support the neuroprotective, neurotrophic and neurotropic actions of neurotrophins, in both PNS and CNS pathology and clinical trials have been initiated. Nerve growth factor (NGF) is the archetype neurotrophic factor. In addition to NGF, this family consists of three known factors, brain-derived neurotrophic factor (BDNF), neurotrophic factor-3 (NT-3) and NT-4. Extensive published research has characterized modes of actions of these factors in the central as well as in the peripheral nervous system mediated via a low-affinity receptor (p75) and three specific high-affinity receptors (trkA, trkB and trkC). In development, they induce neuronal and glial cell differentiation and proliferation, contribute in neuronal wiring by controlling nerve terminal growth, modulation of axonal response to guidance cues, laydown of projections, functional connectivity, target innervation and synapse regulation. After lesion, they come to rescue, by enhancing neuronal survival and reducing apoptosis, preventing axonal die-back and mediating regrowth and sprouting, inducing glial cell proliferation, reinstating myelination of regrowing axons, and by triggering the local expression of other neurotrophins and neurotrophic factors to synergize with them, all to the advantage of regeneration and repair. Furthermore, as the regenerating axon makes contact with glial cells more trophic factors are secreted by the latter, e.g. CNTF by Schwann cells. Thus, the neurotrophins touch off a chain of axon-promotive events effecting regeneration per se and by proxy. Collectively, their beneficial role in axonal regeneration cannot be overstated. Moreover, they are all homodimers, a fact in favor of amphibody manufacture. As is explained in the technical chapter, amphibodies will be produced as fusion proteins, so in this particular case they will spontaneously dimerize, leading to one end carrying the anti-inhibitory moiety, while a fully functional neurotrophin will jut out at the other end. Anyone of the NGF family members could be used to be incorporated in the amphibody, as whole molecule or as the active part only.

NGF e.g.
leads to excessive axon outgrowth/sprouting from sympathetic neurons
mediates remodelling of axon collaterals
effects neurite elongation from axotomized septal-basal cholinergic forebrain neurons
enhances the regeneration of populations of dorsal root ganglia (DRG) axons

BDNF e.g.
prevents axotomy-induced death of corticospinal neurons
enhances survival and regeneration of injured spinal motoneurons
rescues sensory neurons in the neonate following axotomy
promotes survival and sprouting of serotoninergic axons in brain
prevents lesion-induced axonal die-back in optic nerve
induces myelination of regenerating axons in experimental spinal cord injury (SCI)
regulates synaptic innervation density
improves outcome after peripheral nerve transection
enhance retinal ganglion cells (RGC) survival and stimulate axonal branching from regenerating RGC
promotes supraspinal axonal regeneration into Schwann cell grafts in SCI

NT-3 e.g.
prevents axotomy-induced death of corticospinal neurons
promotes corticospinal growth and functional recovery in exp. SCI
rescue sensory neurons in the neonate following axotomy
induces myelination of regenerating axons in experimental SCI
enhances peripheral nerve regeneration after local delivery

NT-4 e.g.
promotes survival of corticospinal motor neurons in neonate
exerts neurotrophic influences on injured spinal motoneurons
enhances retinal ganglion cell (RGC) survival and stimulate axonal branching from regenerating RGC
maintains the cholinergic phenotype of axotomized septal neurons

In this context, see e.g. Levi-Montalcini R, Angeletti P U (1968) Physiolo. Rev 48:534-569; Barde Y A. The nerve growth factor family, Prog Growth Factor Res. 1990; 2(4):237-48. Maisonpierre P C, et al.: Human and rat brain-derived neurotrophic factor and neurotrophin-3: Gene structures, distributions, and chromosomal localizations, Genomics 1991 July; 10(3):558-68; Friedman W J, et al. Transient and persistent expression of NT-3/HDNF mRNA in the rat brain during postnatal development, J Neurosci. 1991 June; 11(6):1577-84; Ip NY, et al. Mammalian neurotrophin-4: structure, chromosomal localization, tissue distribution, and receptor specificity, Proc Natl Acad Sci USA. 1992 Apr. 1; 89(7):3060-4; Ebendal T. Function and evolution in the NGF family and its receptors. J Neurosci Res. 1992 August; 32(4):461-70; Thoenen H, et al. Neurotrophic factors and neuronal death. Ciba Found Symp. 1987; 126:82-95; Kromer L F. Combrooks C J.: Identification of trophic factors and transplanted cellular environments that promote CNS axonal regeneration, in Annals of the New York Academy of Sciences. 495:207-24, 1987. von Bartheld C S.: Neurotrophins in the developing and regenerating visual system, in Histology & Histopathology 13(2):437-59, 1998 April.

### (ii) GDNF-Subfamily

The GDNF family is a subfamily of neurotrophic factors in the TGF-β superfamily. This subfamily consists of four known ligands, GDNF, neurturin (NTN), persephin (PSP) and artemin (ART). They exert their effects through a dual-component receptor consisting of binding proteins with ligand specificity termed GFRa1-4 and a signal-transducing protein called Ret. All four GDNF family ligands are relevant amphibody components. Results from published studies using the same approaches as for the NGF families of ligands and receptors suggest usefulness of amphibodies carrying GDNF-type ligands for regeneration of multiple systems in the central nervous system including the nigrostriatal system, motor systems and sensory systems. Like NGF family ligands, GDNF family ligands also occur as dimers enabling amphibody dimerization as for amphibodies carrying an NGF family ligand.

GDNF e.g.
increases long-term survival of axotomized corticospinal neurons
promote survival of corticospinal neurons in neonate
enhances survival of spinal motoneurons after injury
increases long-term survival of facial motoneurons after axotomy
is a neurotrophic factor for sensory neurons
mediates survival of axotomized mesencephalic dopaminergic neurons
sustains axotonized basal forebrain cholinergic neurons
increases survival of RGC after axotomy
protects against ischaemia-induced injury in cerebral cortex
promotes survival and morphologic differentiation of Purkinje cells

Neurturin e.g.
protects midbrain dopaminergic neurons after axotomy

In this context, see e.g. Unsicker K.: GDNF: A cytokine at the interface of TGF-betas and neurotrophins, in Cell & Tissue Research, 286(2):175-8, 1996 November; Henderson C E. Phillips H S. Pollock R A. Davies A M. Lemeulle C. Armanini M. Simmons L. Moffet B. Vandlen R A. Simpson L C [corrected to Simmons L]. et al.: GDNF: A potent survival factor for motoneurons present in peripheral nerve and muscle. Science. 266(5187):1062-4, 1994 Nov. 11; Lin L F. Doherty D H. Lile J D. Bektesh S. Collins F.: GDNF. A glial cell line-derived neurotrophic factor for midbrain dopaminergic neurons [see comments]. In Science. 260(5111):1130-2, 1993 May 21; Kotzbauer P T, et al.: Neurturin, a relative of glial-cell-line-derived neurotrophicfactor, in Nature. 1996 Dec. 5; 384(6608):467-70; Heuckeroth R O, et al. Neurturin, a novel neurotrophic factor, is localized to mouse chromosome 17 and human chromosome 19p13.3. in Genomics. 1997 Aug. 15; 44(1):137-40 Milbrandt J. de Sauvage F J. Fahrner T J. Baloh R H. Leitner M L. Tansey M G. Lampe P A. Heuckeroth R O. Kotzbauer P T. Simburger K S. Golden J P. Davies J A. Vejsada R. Kato A C. Hynes M. Sherman D. Nishimura M. Wang L C. Vandlen R. Moffat B. Klein R D. Poulsen K. Gray C. Garces A. Johnson E M Jr. et al.: Persephin, a novel neurotrophic factor related to GDNF and neurturin. in Neuron. 20(2):245-53, 1998. Baloh R H. Tansey M G. Lampe P A. Fahrner T J. Enomoto H. Simburger K S. Leitner M L. Araki T. Johnson E M Jr. Milbrandt J.: Artemin, a novel member of the GDNF ligand family, supports peripheral and central neurons and signals through the GFRalpha3-RET receptor complex. Neuron. 21(6):1291-302, 1998 December.

(iii) Neurokines (Neuropoietic Cytokines

This is a distinct family of trophic factors, whose actions are not confined to the nervous system, as is obvious from their hybrid denomination (LIF is capable of regulating hætmatopoiesis and T-cell maturation and IL-6 is involved in the non-specific inflammatory response as an acute phase protein). They share similar structure, bind to receptor subunits of various degrees of homology and their receptor complexes contain an identical signal transducing subunit, gp130. As possible components for amphibodies, the emphasis is on CNTF and LIF that have extensively documented neurotrophic and neuroprotective actions in development and injury. In vitro, in vivo and gene targeting studies have shown that these molecules regulate neuronal and glial gene expression, induce neuronal and glial progenitor differentiation, support the survival of many types of neurons (sympathetic, sensory, motor) and regulate dendritic growth in culture, mediate induction of neuropeptides after axotomy, protect various types of neurons after injury in vivo (motor, sensory) in CNS and PNS, slow down neuronal degeneration and prevent neuronal apoptosis, and as lesion factors expressed by neurons or glial cells after axotomy mediate axonal rescue, sprouting and regeneration. For its part CT-1 is present in embryonic muscle and supports long-term survival of spinal motoneurons. They also show synergy with other neurotrophic factors from the NGF and the GDNF family.

In this context, see e.g. Watters D J, et al. [See Related Articles]: Purification of a ciliary neurotrophic factor from bovine heart, in J Neurochem. 1987 September; 49(3):705-13; Sendtner M. Gotz R. Holtmann B. Thoenen H.: Endogenous ciliary neurotrophicfactor is a lesion factor for axotomized motoneurons in adult mice, in Journal of Neuroscience. 17(18):6999-7006, 1997 Sep. 15; Smith, G M. Rabinovsky E D. McManaman J L. Shine H D.: Temporal and spatial expression of ciliary neurotrophic factor after peripheral nerve injury. in Experimental Neurology. 121(2):239-47, 1993 June; Manthorpe M, et al.: Purification of adult rat sciatic nerve ciliary neuronotrophic factor. in Brain Res. 1986 Mar. 5; 367(1-2):282-6; Patterson P H. The emerging neuropoietic cytokine family: first CDF/LIF, CNTF and IL-6; next ONC, MGF, GCSF? Curr Opin Neurobiol. 1992 February; 2(1):94-7; Rocklin R E. Products of activated lymphocytes: leukocyte inhibitory factor (LIF) distinct from migration inhibitory factor (MIF). J Immunol 1974 April; 112(4):1461-6; Bendtzen K. Some physicochemical properties of human leucocyte migration inhibitoryfactor (LIF). Acta Pathol Microbiol Scand [C]. 1976 December; 84C(6):471-6; Taupin J L, et al. Leukemia inhibitory factor: part of a large ingathering family. Int Rev Immunol. 1998; 16(3-4):397-426; Pennica D, et al. Expression cloning of cardiotrophin 1, a cytokine that induces cardiac myocyte hypertrophy. Proc Natl Acad Sci USA. 1995 Feb. 14; 92(4):1142-6; Peters M, et al. A new hepatocyte stimulating factor: cardiotrophin-1 (CT-1). FEBS Lett. 1995 Sep. 25; 372(2-3):177-80; Pennica D, et al. Cardiotrophin-1. Biological activities and binding to the leukemia inhibitory factor receptor/gp130 signaling complex. J Biol Chem. 1995 May 5; 270(18):10915-22; Pennica D. Arce V. Swanson T A. Vejsada R. Pollock R A. Armanini M. Dudley K. Phillips H S. Rosenthal A. Kato A C. Henderson C E.: Cardiotrophin-1, a cytokine present in embryonic muscle, supports long-term survival of spinal motoneurons, in Neuron. 17(1):63-74, 1996 July.

### (iv) Fibroblast Growth Factor Family (FGF Family)

The FGF family is another separate large family of trophic factors again with more actions than ascribed to them by their name. At least 8 comprise this family but two of them, namely acidic and basic FGF, stand out as neurotrophic for neurons from multiple CNS regions and neuroprotective in CNS and PNS with enough in vitro/in vivo studies demonstrating regulation of postinjury neuronal survival, axonal growth, neurite extension on the ECM, and a clear-cut positive role for regeneration. They exert these effects by activating the FGF receptor on neurons, a non-CAM Ig superfamily member, the same effector unit that Ig CAMs like NCAM, L1 and N-cadherin interact with on the axonal membrane to promote contact-dependent axonal growth. It is also obvious why CAMs make excellent candidates for the pro-end of amphibodies as will be discussed below. Also, basic-FGF in synergy with IL-1, a cytokine produced locally after injury by blood cells strongly enhances axonal growth.

In the context of FGF, see e.g. Cuevas P, Gimenez-Gallego G: Role of FGFs in neural trauma, in Neurol Res 1997 June; 19(3):254-6

### (v) Hepatocyte Growth Factor (HGF)/Scatter Factor

HGF is now known to be a guidance and survival factor in the developing nervous system. Netrins was the only family of diffusible chemoattractants for developing axons before the characterization of a similar role for HGF. It is derived by the limb mesenchyme and thereby guides spinal motor neurons to their targets in the limb. Moreover, it has a role in later stages as target organ (muscle) derived neurotrophic factor for motor neurons and has neurotrophic effects on CNS neurons in culture.

In the context of HGF, see e.g. Ebens A. Brose K. Leonardo E D. Hanson M G Jr. Bladt F. Birchmeier C. Barres B A. Tessier-Lavigne M.: Hepatocyte growth factor/scatter factor is an axonal chemoattractant and a neurotrophic factor for spinal motor neurons. in Neuron. 17(6):1157-72, 1996 December.

### (vi) Other Factors

Members outside the GDNF subfamily but of the same superfamily have been reported to exert benefit for regenerating axons. TGF-β1 is an injury-related growth factor with a neuroprotective effect in the CNS and has been shown to regulate axon/Schwann interaction and to induce expression of other trophic factors after axotomy, e.g. LIF. TGF-β3 has been reported to promote motoneuron survival.

### 2. Construction of Amphibodies

### 2.1 Methods

### 2.1.1 Antibodies; Introduction

Antibodies have been evolved by nature as molecules that have a common general structure, but can vary in certain crucial parts allowing them to bind to different molecules. Thus, a repertoire of antibodies exist in any organism with a capacity to produce such molecules. Strictly speaking, the word immunoglobulin (Ig) is used as a general term for this type of molecules, while antibodies are used for such molecules when the molecule they bind to is known; this “target” molecule is defined as the “antigen”. Most often, however, “immunoglobulin” and “antibody” are used synonymously. In the body, immunoglobulins are produced by B-lymphocytes, a kind of white blood cells. The antibody system has been devised to have the potential to produce binding molecules to practically any upcoming target. This is accomplished by an intricate combination of reshuffling of gene fragments, imprecise joining of such fragments, additions of nucleotides to existing gene fragments, and by occasional point mutations in the immunoglobulin genes. It is estimated that the total potential of different Ig molecules in this way may total 10¹¹-10¹⁵ different specificities, i.e. binding molecules of different reactivity.

The antibody molecule consists of a limited number of globular modules, the immunoglobulin domain, that are fused together in “chains” of polypeptides. This domain has a conserved structural motif, and consists of approximately 110 amino acid residues. The most common immunoglobulin in human serum, the IgG molecule, consists of two identical “heavy” chains comprising four domains, and two identical “light” chains of two domains each. In the N-terminal of these chains sits the variable domain; it is the combination of variable domains of the heavy and light chains that determines the specificity of the antibody, i.e. what binding capacity it will have and thus what target it will bind to. Indeed, for any given B-lymphocyte clone, the actual sequence of the variable domains may be strictly individual, and may thus serve as a “fingerprint” for the actual B-lymphocyte clone.

The other domains are conserved, and their polypeptide sequence is thus shared between immunoglobulins stemming from different B-lymphocyte clones.

Differences in the conserved regions, however, give rise to the division of immunoglobulins into classes and subclasses of Ig. Also, the conserved parts interact with other cells and molecules of the bodies defence system, and are thus referred to as the effector function of the Ig molecule. Accordingly, the effector functions may vary depending of the class or subclass of the molecule.

The capacity of the immune system to create binding molecules to any given target molecule has for decades been harnessed by the biomedical scientists, most notably by immunising an animal with a certain substance, and then isolating a collection of Ig molecules directed to that substance from the blood of the animal. Modern biomedicine, however, have expanded on this in that we now can generate single antibody molecules of predetermined specificity by isolating the B-lymphocyte producing the antibody, and let this cell grow perpetually giving rise to a clone of daughter cells. This is called hybridoma technology, as the B-cells are made immortalised by the fusion of the B-lymphocyte with a continuously growing cancer cell (myeloma) (see below, section 2.1.2). A more recent approach is to isolate and rescue the genes for a single antibody molecule of a particular specificity; these genes are subsequently arranged in a vector for expression of the antibody in various host cells (even bacteria) (see below, section 2.1.3). The genetic approach does allow for additional manipulations of the antibody molecule, such that unrelated molecules may be attached to one of the Ig chains (section 2.1.5).

The binding of antibodies to a target may invoke different effects, depending on the conditions and the target. Thus, sheer binding may inhibit the function of the target molecule, eg. of a toxin that won't be able to react with its normal receptor or attachment mechanism. Antibody binding may also induce activation, e.g. binding to a receptor molecule on the cell surface. Also, if the antibody is linked to a “label”, a molecular entity that can be detected by X-ray or by other means, antibodies can be used to image processes in the body. Certainly, such a concept may also be used for therapeutic interventions, e.g. the antibody can be used to deliver a drug or toxin to a certain location in the body (e.g. tumour).

Recently, bioorganic chemistry, in combination with protein engineering has allowed the creation of antibodies that not only bind but also change the target molecule by catalysis, i.e. the antibody works as an enzyme.

The present invention expands on some of the aspects of antibody technology mentioned above. Thus, a more detailed description will follow on particular aspects related to the invention.

### 2.1.2 Hybridoma Methods

Rescuing of antibody producing B-lymphocytes by immortalization of these cells was first accomplished by Köhler and Milstein (published in 1975). Mice are immunised with the antigen to which antibodies are sought. The result of the immunisation is monitored by assessing the occurrence of the required antibodies in the blood of the animal. When a sufficient concentration (titre) of antibody has been achieved, the animals are sacrificed and the spleen rescued. The white blood cells harboured in the spleen are isolated by cutting the organ into small pieces, and subsequent gentle manipulation of these pieces in order to release the white blood cells in the spleen. The cells are diluted in a large volume of physiological salt solution, collected by gravity or centrifugation, and then fused to a uniform collection of immortalised cells, usually a murine myeloma that has been manipulated to lack certain enzymes necessary for growth in presence of molecules blocking the natural synthesis of nucelotides. The fusion between the cell types is accomplished either by chemical means, using polyethylene glycol, or by biological means, using Sendai virus. Once the cells have fused, the growth conditions will be composed such that the natural way to synthesise nucleotides will be blocked. Only the spleen cells carry the capacity to use an alternate pathway. However, fusions of myeloma and spleen cells will also survive; spleen cells not having fused to myeloma will die after a limited number of cell divisions, as all normal cells have a limited life span. The fused cells, however, will benefit from the immortalised property of the myeloma, and will propagate perpetually. Cells producing antibodies will release these into the medium; detection of Ig in supernatants from the cell cultures will thus be performed, and positive cultures will then be diluted such that only single cells will give rise to subsequently propagated colonies of antibody producing, immortalised cells.

### 2.1.3 Genetic Methods

Advances in gene technology has made possible the generation of antibody molecules utilizing the genetic material coding for antibodies. Genetic material coding for antibodies can be rescued from antibody producing cells, i.e. from B lymphocytes directly, or from already established hybridomas producing a monoclonal antibody. The immunoglobulin DNA can be isolated as genomic DNA or as RNA that is converted into cDNA. If the Ig genes obtained stem from already established hybridomas, expression of the genes after having cloned them into suitable vectors should result in an antibody with predetermined specificity, i.e. a molecule with the same binding characteristics as the antibody molecules secreted from the hybridoma.

In contrast, if the Ig genetic material is obtained from a population of B lymphocytes, a library of different antibodies is constructed, and a selection process allowing the selection of antibodies having the desired binding characteristics (e.g. binding to a certain target) has to be included in the work. In practice, such a library is cloned into vectors allowing the display of each protein linked to the gene for that same protein in a defined cell, particle or multi-molecular complex (pro- or eucaryotic cells, virus, ribosome complexes etc.) Such display systems facilitate the rescuing of the genes coding for antibodies with desired binding properties, as the cell/particle/molecular complex is selected by binding to the target, and the genes may subsequently be rescued from the bound cell/particle/complex, while genes for antibodies that do not bind, and hence are of no interest as reagents, will not be rescued.

Once the genes of a certain antibody has been obtained as defined genetic material, it can be analyzed, manipulated and modified in a large number of ways, e.g. for nucleic acid sequencing, introduced into pro- and eucaryotic cells for the expression of the whole antibody or fragments thereof, fused to other genes for production of fusion proteins etc. etc.

For genomic and cDNA cloning from hybridomas standard cloning protocols can be used (Sambrook et al, 1989). Once obtained, cloning into vectors and recombinant expression of antibodies has been detailed in Morrison 1995. For cloning of antibodies from antibody libraries, please see Huse et al. 1989, Clackson et al 1991, Samuelsson et al 1996, Rader & Barbas 1997, and references therein. Obviously as for all recombinantly expressed proteins, subsequent purification of the expressed material is needed.

### 2.1.4 Fusion Proteins

Fusion proteins are simply two naturally single proteins irreversibly fused together, either by chemical or, more often in contemporary biomedicine, genetic means. The resulting protein, the so called fusion protein, has the combined properties of the two components. For example, if a ligand for a cell surface receptor is fused with a toxin, then the fusion protein has both these properties, i.e. will bind to the receptor and have a toxic effect. Very likely in this example, the function of the cell carrying the receptor will be severely affected as an effect of the toxin, while other cells will go unaffected as the fusion protein will not bind to them. The use of fusion proteins have been expanding over the last 10-15 years, both in biotechnology, medical therapy and diagnostics (Uhlén et al. 1992, Murphy et al. 1987, Haber 1992, Murray & Dahl 1997, Ford et al. 1991). Production of fusion proteins by chemical means requires that the two components first are produced and purified, and subsequently coupled together by the creation of covalent bonds between them, as described in the section “Chemical methods for the generation of fusion proteins”. If the fusion protein is generated by genetic means, the genes (genomic or cDNA) are linked together in a uni cistronic fashion often with a linker region places between the two main gene components. One way to accomplish such a splicing of genetic material is by the use of overlapping polymerase chain reaction, as depicted elsewhere in this application under the heading “Experimentals”. In either case, purification of the resulting fusion protein is most often necessary. As a special case, a dual affinity purification may be applied to fusion proteins, selecting only those molecules where both the main components are included. In addition, it should be noted that fusion proteins may consist of more than two separate molecular components, e.g. a fusion protein consisting of a receptor ligand-toxin-fluorescent molecule can easily be conceived.

### 2.2 Putative Design of Amphibodies

The design and construction of the coding nucleic acid parts of a conceptual amphibody can be described as follows. The concept implies the incorporation of three major parts: the antibody, the neurotrophic factor, and a linker in between the two former ones (FIG. 4). In addition, a dimerizing domain may be added if necessary for the proper function of the neurotrophic factor.

### 2.2.1 The Antibody Portion

As antibody molecule, a single chain construct of an antibody fragment will preferably be utilised, as this facilitate the inclusion of the antibody molecule genes in a fusion protein of mentioned type. Such single chain constructs include both variable domains (VH and VL) linked via a peptide linker to form a continuos antigen binding molecule, a so called scFv. The order of the genetic elements may be either VH-linker-VL, or vice versa: VL-linker-VH. For certain purposes, one being stability of the molecule, the constant domain of the light chain may be included, giving a whole Ig light chain linked to the VH region (so called scVH-LC format).

### 2.2.2 The Linker Region

The linker region of the amphibody should allow the different parts of the molecule to assume proper functional conformation though fused with the other proteins. Several approaches to achieve this has been reported, very often in the construction of single chain antibodies as mentioned above. In nature, similar properties are often required within existing molecules, i.e. several natural proteins have been evolved by combining different peptide motifs, with “linker” regions in between. Thus, a common linker motif found in natural proteins is GlyGlyGlySer (SEQ ID NO: 1) (or GlyGlySer). It can be shown by energy maximum/minima calculations that such motifs allow a very large degree of freedom that facilitate the different fusion protein parts to retain the functions of the original proteins. Based on experiences with linker regions for the construction of single chain antibodies, we suggest that the linker between the antibody and the neurotrophic molecule part should be (GGGGS)×3 (SEQ ID NO: 2). However, the linker may need to be optimized for the actual proteins it is supposed to fuse together. This may be achieved by randomizing several of the amino acid residue positions within the linker, and then select the variant with the better functional properties.

### 2.2.3 The Neurotrophic Portion

As mentioned elsewhere in this application, a variety of molecules may be used for this purpose. Here, only aspects related to its inclusion in a fusion protein will be mentioned. Most importantly, its free movement from the antibody portion has to be assured by utilizing a functional linker region (see above).

### 2.2.4 Dimerization Domain

For certain neurotrophic molecules, their proper function is only obtained when the molecule is presented as a dimer to the nerve cell. Often, such molecules have peptide motifs or regions that induce an interaction such that a dimerization is performed. Should such interacting domains not be present in the neurotrophic factor chosen, or the dimerization capacity of the normal molecule hampered as a result of the proximity to the other portions of the fusion protein, a dimerization domain may be added into our construct. A natural motif used for this purpose is the “leucine zipper” motif, often found in transcription factors. Dimerization is promoted by the formation of a “coiled-coil”, i.e. a dimer of parallel helices. The transcripiton factors jun and fos consist of leucine zipper domains that preferentially form heterologous dimers (jun-fos rather than jun-jun or fos-fos). Thus, if dimerization needs to be engineered into our amphibodies, two variants of the gene construct may be produced, one with the jun domain in the 3′ end, the other identical except for that the jun domain will be replaced with the gene for the fos domain. The molecules will be expressed/produced separately, and let to dimerize in vivo.

### 2.3 Chemical Methods

The construction of the genes coding for the amphibodies will be performed by overlapping PCR. In this technique, the different portions of the gene (e.g. A and B) to be assembled will be amplified by PCR using primers that include sequence motifs allowing the hybridization of PCR products that should d be assembled adjacent to each other. Thus, PCR products A′ and B′ are obtained in a first step. In a second PCR reaction, the DNA strands of the two PCR products A′ and B′ to be assembled in the arrangement A-B are dissociated, and when annealed, a proportion of the strands will hybridize in a cross fashion: sense strand of A′ with anti-sense strand of B′ and v.v. In the polymerization step, the extended product A+B will be produced, facilitated by the addition of primers hybridising to the 5′ end of A and to the 3′ end of B (FIG. 4).

This procedure of overlapping may be repeated in succession, adding on genes to an already existing assembly, i.e. genes A and B assembled into A+B may be combined with gene C, giving A+B+C. In this way, we may construct different combinations of single chain antibody genes with different linkers and neurotrophic molecules, with or without dimerization domains added. Preferably, DNA polymerases with very low misincorporation rate should be utilized, as the number of PCR cycles may become substantial in order to assemble several genes in this way.

### 2.4 EXAMPLE 1

### Construction of a Fusion Protein Consisting of NT3 and an Antibody

The design of a tentative fusion protein consisting of NT-3 and an Fab version of an antibody to NOGO have the following outline:

N-terminal-NT-3-linker-antibody heavy chain (Fd fragment)-stop-antibody light chain.

Using a flexible linker between the NT-3 portion and the Fd chain, it is assumed that both will be fully functional. Also, the NT-3 is provided as full length cDNA, i.e. in order to be subject to processing inside a eucaryotic cell, and only present the mature protein (the COOH part of the NT-3 cDNA). The antibody light chain have intrinsic affinity for the Fd molecule, and has been show to interact correctly in earlier attempts to express Fab molecules in various host cells.

Cloned NT-3 and a cloned Fab antibody, IN-1, to NOGO, were obtained. They were combined using a sequence of point mutations and PCR amplifications as follows:

1. The existing stop codon in NT-3 was mutagenised using hybridising primer (881-S (SEQ ID NO: 10) and 848-AS (SEQ ID NO: 11)). A mutagenesis kit (Stratagene, Prod No. 200518-5) was purchased and used for the reactions. 9 out of 10 clones analysed carried the introduced mutation.

2. Nucleic sequencing was performed on the 5′ end of the NT-3 cDNA, in order to be able to design PCR primers correctly; the primers used for sequencing were 774-S (SEQ ID NO: 9) and 169-AS (SEQ ID NO: 8).

3. The Fd chain of IN-1 was amplified in a two step PCR, in order to extend the 5′ end of the resulting product, so that it contained several restiction endonuclease sites, allowing the later insertion of NT-3 5′of the Fd cDNA, as well as a linker region between the insertion site for NT-3 and the antibody chain cDNA. The sequence of IN-1 was located in Genebank. In the first PCR experiment, the primers AB-239-S (SEQ ID NO: 4) and AB-912-AS (SEQ ID NO: 5) (5′ and 3 primers, respectively) were used.

In the second PCR, utilising a fraction of the PCR product from the first reactionas template, the primers AB-176-S and AB-912-AS were used. Primer AB-23 9-S (SEQ ID NO: 4) carries a XhoI restriction site, and the second 5′ primer AB-176-S (SEQ ID NO: 3) carries HindIII, SalI, and AgeI sites.

4. The light chain of IN-1 was PCR amplified using primers AB-996-S (SEQ ID NO: 6) and AB-1647-AS (SEQ ID NO: 7). The latter primer contains an XbaI site.

5. The PCR amplifications all worked well, and two final PCR products were obtained. The ends were digested with HindIII and NheI (the Fd PCR product from step 3), and Ecl136II+XbaI (light chain). They were gel purified in agarose gel.

6. The vector pcHCLC was prepared for subsequent ligation of the Fd and LC PCR products by digestion with
i.) HindIII+NheI, and
ii.) ii.) HpaI +XbaI, for the insertion of Fd and LC PCR products, respectively. The linearised vector DNA was obtained by gel purification.

7. The digested LC PCR product (from step 4 and 5) was successfully ligated into pcHCLC (from step 6 i.).

8. NT-3 cDNA was PCR amplified from the mutagenised version obtained in step 1 using primers 96-S (SEQ ID NO: 12) and 848-AS (SEQ ID NO: 11). The PCR product was digested with XhoI and AgeI, and gel purified.

Then, the Fd PCR product is ligated into pcHCLC already containing the IN light chain DNA (from step 7) followed by digestion with SalI and AgeI and subsequent gel purification of the plasmid obtained above. The digested PCR product obtained in step 8 is ligated into the linearised plasmid DNA obtained as described above. The plasmid so obtained is used to transfect mammalian cells and an assay for expression of the recombinant protein is performed.

Thus, cells expressing a fusion protein according to the invention are obtained. Growth conditions, methods of purification etc are conventional techniques well known to the skilled in this field.

Primers used in Example 1:

AB 176-S

(SEQ ID NO: 3)

AGA AAA GTG AAA TGG AAG CTT CGA CAA AAT TCT GTC
GAC CGA GGG CAA AAA ACC GGT GGG GGA GGT TCC GGG
GGT GGA GGG
AB 239-S

(SEQ ID NO: 4)

GGG AGG TTC CGG GGG TGG AGG GTC AGG GGG TGG TGG
CTC TGG CGG AGG GGG GTC CGA AGT TAA ACT GCT CGA
GTC AGG GCC TGG GCT GGT AAG GCC TGG G
AB 912-AS

(SEQ ID NO: 5)

TTC TGC TAG CTT ATT AAT GGT GAT G
AB 996-S

(SEQ ID NO: 6)

AAA AGC CGA CAT CGA GCT CAC C
AB 1647-AS

(SEQ ID NO: 7)

GCA GCA TCA GGA CCT CTA GAA CTA ACA CTC
169-AS

(SEQ ID NO: 8)

GGG AAT TGA GAG AGT CTT CTG G
774-S

(SEQ ID NO: 9)

GCA AAC CTA CGT CCG AGC ACT G
881-S

(SEQ ID NO: 10)

GAA AAA TCG GAA GAA CCG GTA TTG GCA TCT GTC C
848-AS

(SEQ ID NO: 11)

GGA CAG ATG CCA ATA CCG GTT CTT CCG ATT TTT C
96-S

(SEQ ID NO: 12)

GAT CTT ACA GGT GAA CCT CGA GAT GTC C

1. A neuromodulator molecule comprised of at least two components, wherein
a first component is capable of binding to a target and suppressing, or essentially neutralising, a neurite growth inhibitory effect of said target; and
a second component is capable of stimulating neurite growth and/or regeneration.

2. A neuromodulator according to claim 1, wherein said two components are separated from each other by a linker element to assume a functional conformation.

3. A neuromodulator according to claim l, wherein the target is a glial cell, a neuron, a fibroblast, a blood cell or an extracellular matrix component, which provides a neurite growth inhibitory effect by expressing a specific neurite growth inhibitory molecule.

4. A neuromodulator according to claim 1, wherein said neurite growth inhibitory effect is provided by at least one naturally occurring neurite-growth inhibitor selected from the group consisted of: NOGO; a myelin associated glycoprotein (MAG); a proteoglycan; a Sem-receptor; and a member of any one of the families of semaphorins; tenascins, netrins and Eph and ephrins.

5. A neuromodulator according to claim 4, wherein said first component is IN-1 and said inhibitory factor is NOGO.

6. A neuromodulator according to claim 1, wherein said second component is a neurotropic molecule.

7. A neuromodulator according to claim 6, wherein said neurotrophic molecule is a cell adhesion molecule (CAM) selected from the group consisting of an immunoglobulin superfamily CAM, a cadherin and an integrin, or a functional fragment thereof.

8. A neuromodulator according to claim 6, wherein said neurotropic molecule is an extracellular matrix molecule (ECM).

9. A neuromodulator according to claim 1, wherein said second component is a neurotrophic molecule.

10. A neuromodulator according to claim 9, wherein the neurotrophic molecule is any one selected from the group consisted of members of the neurotrophin family; the GDNF-subfamily; neuropoietic cytokines; fibroblast growth factors (FGF) and hepatocyte growth factor (HGF)

11. A neuromodulator according to claim 10, wherein said second component is NT3 or another neurotrophin, such as BDNF, NGF or NT4.

12. A neuromodulator according to claim 10, wherein said second component is L1.

13. A method of producing a neuromodulator according to claim 1 by recombinant DNA technique, which comprises the steps of
(a) providing a nucleic acid encoding a suitable first component capable of binding to a target and suppressing, or essentially neutralising, a neurite growth inhibitory effect thereof;
(b) providing a nucleic acid encoding a suitable second component capable of stimulating neurite growth and/or regeneration;
(c) fusion of said nucleic acids into a recombinant nucleic acid construct;
(d) providing a vector carrying said construct;
(e) insertion of said vector into a suitable host cell; and
(f) expression of the desired regulator.

14. A method of producing a neuromodulator according to claim 1 by chemical fusion of a first and a second component, which method comprises the steps of
(a) providing a first component capable of binding to a target and suppressing, or essentially neutralising, a neurite growth inhibitory effect thereof;
(b) providing a second component capable of stimulating neurite growth and/or regeneration;
(c) chemical fusion of said components with a suitable reagent to produce the desired amphibody.

15. A method according to claim 14, wherein said components are selected from the group consisted of proteins, polypeptides, peptides and carbohydrates.

16. A method according to claim 14, wherein in step (c), the ratio between the first and the second component is about 1:1.

17. A method according to claim 13, which method also includes a step for purification of the desired modulator from any undesired matter.

18. A vector comprising nucleic acids encoding a neuromodulator according to claim 1.

19. A cell comprising a vector according to claim 18.

20. A pharmaceutical preparation comprising a cell according to claim 19, and preferably comprising a suspension of such cells, together with a pharmaceutically acceptable carrier, which preparation is suitable for use in gene therapy methods.

21. A neuromodulator produced according to the method of claim 13 for use as a medicament.

22. A method of using the neuromodulator according to claim 1 in the manufacture of a medicament for the treatment and/or prevention of spinal cord injury, brain trauma, stroke, retinal and optic nerve lesions, neurodegenerative diseases, neuromuscular diseases, autoimmune diseases of the nervous system, tumors of the central nervous system etc.

23. A pharmaceutical preparation comprising a neuromodulator according to claim 1 together with a pharmaceutically acceptable carrier.

24. A pharmaceutical preparation, which is a cocktail of different neuromodulators according to claim 1 together with a pharmaceutically acceptable carrier.
